(19) United States
(12) Patent Application Publication
(10) Pub. No.: US 2006/0073611 A1
(43) Pub. Date: Apr. 6, 2006

(54) Immunoassay

(76) Inventor: David John Grainger, Cambridge (GB)

Correspondence Address: CLARK & ELBING LLP, 101 FEDERAL STREET, BOSTON, MA, 02110, US

(21) Appl. No.: 10/547,995

(22) PCT Filed: Mar. 10, 2004
(86) PCT No.: PCT/GB04/01016

§ 371 Date: Oct. 28, 2005

(51) Int. Cl. G01N 33/543

(52) U.S. Cl. 436/518

(57) ABSTRACT

The present invention relates to methods of assaying the levels of proteins or antibodies in a test sample. In particular, the present invention relates to a method of determining the relative abundance of a plurality of proteins in a test sample compared to a reference sample, the method comprising: (a) providing a reference sample comprising a plurality of labelled proteins; (b) incubating a plurality of tagged antibodies capable of binding components of the reference sample with (i) a mixture of the labelled reference sample and the test sample and (ii) the reference sample alone, under conditions suitable for the binding of said antibodies to their targets; (c) comparing the amount of labelled protein bound to individual antibody tags in the presence and absence of the test sample.

The present invention relates to methods of assaying the levels of proteins or antibodies in a test sample. More particularly, methods are provided which allow the relative concentration of many proteins in a pair of samples to be rapidly determined. Further methods are provided which generate a profile of the array of antibodies present in a test sample.

### BACKGROUND TO THE INVENTION

Increasingly, scientific advances and technological applications are depending on the capability to measure many different parameters about a complex system, such as a living cell, simultaneously. The first examples to become widely available in biology of such “holistic” analyses came from the introduction of “gene chips” which could analyse the levels of gene expression for many hundreds or thousands of genes simultaneously. This technology, which underpins the field of genomics (the study of the co-ordinate regulation of all the genes in the organism), is now ubiquitous and has brought a number of benefits to science and technology.

However, genomics is not the only “omics”—the term given to branches of sciences devoted to examining the co-regulation of parameters within a complex system. Proteomics is the term given to the study of the regulation of all the proteins present in a cell, tissue or biological sample. Metabonomics is the analogous study of all the non-protein (usually low molecular weight) metabolites, such as sugars and fats, in a cell, tissue or biological sample. Both proteomics and metabonomics have been shown to be useful for diagnosing human diseases much more powerfully that the conventional approach of measuring just a few candidate disease markers (such as measuring cholesterol levels to diagnose the presence of heart disease).

The utility of “omics” approaches to understanding complex systems (such as human beings) is limited by the ease and robustness of the underpinning technology. For example, it was the introduction of commercially available gene-chips that led the current rash of genomics research and technology.

In genomics, the gene array tools currently available are relatively easy to use, although they require certain small and relatively cheap specialist pieces of equipment which need to be installed and maintained. Unfortunately, the results obtained are not particularly robust, with coefficient of variations for repeated measures often exceeding 25%. Such inaccuracy severely hampers the use of gene array technology in many, if not all, applications.

Conversely, in metabonomics the tools currently available (such as NMR and IR spectroscopy or mass spectrometry) are inherently robust, often producing repeated-measures coefficients of variation below 2%. However, they are intrinsically complex technologies requiring not only significant capital investment (an NMR machine, for example, may cost in excess of half a million pounds) but also extensive specialist knowledge to operate in a useful way.

Proteomics currently lies somewhere between these two extremes: the technology is somewhat accessible and somewhat robust. Currently, the approaches to proteomics fall into two broad groups: separation based techniques and whole sample techniques.

Considering the separation-based techniques first, the two most commonly used separation technologies are gel electrophoresis and tandem liquid chromatography. In both cases, the protein mixture is separated into components, which are then analysed by electrospray tandem mass spectrometry to identify the component. These techniques require relatively specialist and capital intensive equipment, and they produce data with repeated measures coefficients of variation down to 10%. Neither technique, however, is well suited to high throughput applications and the amount of data processing required for a single sample is often very large indeed.

The whole sample approach has the advantage of being intrinsically more suited to high throughput applications, such as clinical diagnostics. Unfortunately, the current approaches (of which the best established is the shot gun tandem mass spectrometry approach in which the entire sample is fragmented and then the sequence of each fragment determined) suffer from the inability to detect and quantify any but the most abundant proteins within the sample mixture. For many biological specimens, where the analytes of interest may vary in concentration over 6 orders of magnitude, the current approaches are essentially useless. The number of protein fragments that must be analysed from a human serum specimen in order to sample more than 1% of the constituent proteome is so large as to be impractical. Even the introduction of pre-preparation steps, where the most abundant proteins of all, such as serum albumin, are selectively removed prior to analysis only slightly improve the performance. In principle, such approaches are unlikely ever to provide a rich sampling of the low- and mid-abundance components of the proteome.

Another whole-sample approach is the use of protein-chip (microarray) technology. The principle here is identical to gene chips genomics (which detects the interaction of DNA or RNA in the test sample with a DNA probe on the chip surface). Instead of DNA probes, antibody molecules are coated onto the microarray and the binding of the antigen to the antibody can be quantitated. Such approaches avoid the limitations of other whole sample approaches: like DMI, they can in principle quantitate proteins irrespective of their relative abundance in the test sample. Unfortunately, this approach has a number of limitations—most severe is the inherent lack of quantitative robustness in the microarray detection methodology. The same limitations which reduce the repeatability in micro-array based genomics also prevent the widespread adoption of micro-array based proteomics.

Consequently, there is a need for new proteomic technology which combines all the desirable characteristics of such a technology: it should be a rapid, high throughput approach which avoids the use of technically specialised procedures or capital intensive equipment, and which provides an unbiased sampling of the proteome irrespective of the absolute abundance of the components present, and which is quantitatively robust under routine laboratory conditions.

### SUMMARY OF THE INVENTION

The present invention provides methods which allow the relative concentrations of many proteins in a pair of samples to be rapidly determined. A tagged antibody library is exposed to a mixture of the test sample and the reference sample, where the reference sample has been labelled in some way. For a given antibody, the amount of label that is bound will be inversely proportional to the amount of the cognate antigen present in the test sample. The amount of label bound to each tagged antibody is read in turn to generate a vector describing the relative pattern of protein concentrations in the two samples.

Accordingly, the present invention provides a method of determining the relative abundance of a plurality of proteins in a test sample compared to a reference sample, the method comprising (a) providing a reference sample comprising a plurality of labelled proteins, (b) incubating a plurality of tagged antibodies capable of binding components of the reference sample with (i) a mixture of the labelled reference sample and the test sample and (ii) the reference sample alone, under conditions suitable for the binding of said antibodies to their targets, (c) comparing the amount of labelled protein bound to individual antibody tags in the presence and absence of the test sample.

Methods falling under this embodiment may be useful for proteomics (the science of studying large populations of proteins simultaneously). An example of such a proteomic application would be in clinical diagnostics, whereby measuring the levels of many proteins in a biological specimen simultaneously could be used to make a diagnosis of a disease or condition.

The same principle may also be applied to the profiling of the array of antibodies that are present in a sample, for example the array of antibodies made by different individuals. Such a profile may be diagnostic of the immune status of the individuals from whom the samples were obtained.

The present invention also provides a method of detecting a plurality of immunoglobulins in a test sample, the method comprising (a) providing a plurality of tagged antigens, (b) incubating said tagged antigens of (a) with said test sample, under conditions suitable for the binding of any immunoglobulins present in said test sample to their targets, (c) incubating said mixture of (b) with one or more labelled antibodies capable of binding specifically to immunoglobulins, (d) measuring the amount of labelled antibody bound to each tagged antigen.

The present invention also relates to groups and libraries of antigens, in particular peptides for use in such methods. In particular, the invention provides a mixture of peptides wherein each peptide is of length n amino acids and of the formula:
X₁—X₂—X₃— . . . —Xₙ
wherein:
each X represents an amino acid independently selected from one of a number of groups of amino acids;
each group of amino acids consists of less than 20 different amino acids,
n is the same for all peptides present in the mixture;
all of the following amino acids are present in at least one group: arginine, lysine, histidine, glutamate, aspartate, proline, cysteine, serine, threonine, tryptophan, glycine, alanine, valine, leucine, isoleucine, methionine, asparagine, phenylalanine, tyrosine and glutamine, and
for each peptide in the mixture the amino acid at the same position is selected from the same group.

Also provided is a library comprising a plurality of such mixtures wherein each of said mixtures has the same value for n and the same groups of amino acids apply to all mixtures in the library, wherein (a) no peptide is present in more than one of said mixtures, and/or (b) the mixtures differ by virtue of the fact that the combination of groups chosen to obtain the peptides differs between the mixtures and optionally the library comprises mixtures representing all possible combinations of the groups.

The invention also provides methods for the diagnosis of diseases and other medical conditions. In particular, the invention provides a method of detecting the presence of, or a susceptibility to, a disease or other medical condition comprising:
(i) detecting a plurality of immunoglobulins in a test sample obtained from an individual; and
(ii) comparing the immunoglobulins detected in the sample from said individual with known patterns of immunoglobulins associated with the presence or absence of a disease and thus determining whether said individual has, or is susceptible to said disease.

Also provided is a method of detecting the presence of, or a susceptibility to, a disease or other medical condition comprising:
(i) detecting a plurality of immunoglobulins in test samples obtained from individuals whose disease status is known;
(ii) comparing the immunoglobulins detected between those individuals who are disease sufferers and those who are not and identifying any patterns associated with the presence or absence of the disease;
(iii) detecting a plurality of immunoglobulins in a test sample obtained from an individual by the same method used in part (i); and
(iv) comparing the immunoglobulins detected in the sample from said individual with the patterns identified in step (ii) and thus determining whether said individual has, or is susceptible to said disease.

The invention further provides kits suitable for use in the immunoassay methods of the invention. In particular, a kit is provided comprising
(i) a plurality of antigens or mixtures of antigens, wherein each antigen or mixture of antigens comprises a tag; and
(ii) one or more labelled antibodies capable of specifically binding to immunoglobulins.

In a further aspect, the invention provides a method of reducing the redundancy and bias of an antibody-expressing phage library comprising:
(a) providing two surfaces to which a sample of antigens is bound wherein said antigens are bound to the second surface at a higher density than to the first surface;
(b) exposing a phage display library to a first surface of (a) under conditions suitable for antibody binding and selecting phage bound to said surface; (c) exposing said selected phage of (b) to a second surface of (a) under conditions suitable for antibody binding and selecting phage not bound to said surface;
(d) optionally further selecting said phage of (c) according to steps (b) and (c) one or more times;
   thereby obtaining a library of antibody-expressing phage which has reduced redundancy and/or bias characteristics compared with the original library. An antibody library obtained by such a method may be tagged and used in a screening method of the invention.

### BRIEF DESCRIPTION OF THE FIGURES

FIG. 1: Schematic representation of two embodiments of the invention.

A: A library of antibodies against the proteins of interest is constructed. Such a library should be highly representative of the proteins in the sample under test, and have a low degree of redundancy (so that antibodies against the same protein do not occur more than a small number of times in total in the whole library). This library is then tagged using one of a range of commercially available tagging technologies, such as the SmartBead platform that uses aluminium barcode tags made by semiconductor fabrication technology.

The specimen under test is then mixed with a reference specimen which has been labelled with a suitable label (for example a fluorescent marker). The mixture of test and reference samples is then incubated with the tagged antibody library and the amount of labelled protein that binds to its cognate antibody is influenced by the amount of the same protein present in the unlabelled test sample. If the protein level is higher in the test sample, the amount of label bound to the tagged antibody is decreased, while if the protein level is lower in the test sample, the amount of label bound to the tagged antibody is increased.

The library is then passed through a laboratory flow cytometer that can read both the tag and barcode and quantify the amount of fluorescence label bound. This approach may be capable of generating up to 1 million datapoints in 15 minutes. Provided that the redundancy of the antibody library is very low, this translates into a relative measure of the level of hundreds of thousands of proteins.

The protein profile that is generated (a vector containing many numbers representing the relative levels of fluorescence bound to each of the tagged antibodies) can be analysed by conventional megavariate pattern recognition methods and provide a protein “fingerprint” for the sample class under study.

B: An antigen library is generated and coupled to the tags, analogous to those in A. This library is then exposed to the test sample of human serum and antibodies in the serum bind to the library of antigens. Any bound human immunoglobulin is then detected by addition of a standardised solution of anti-Ig antibodies labelled with different fluorophores. For example, by using anti-IgG labelled with the green fluorophore fluorescein and anti-IgM labelled with the red fluorophore rhodamine it is possible to simultaneously quantify the amount of each immunoglobulin subclass which binds to each antigen in turn.

FIG. 2: A chromatogram of a typical reference sample after labelling the protein with fluorescein isothiocyanate, as described in the text. The labelled sample is applied to a Sephadex G25 column and the eluate is monitored at 280 nm (A280) and 450 nm (A450). The labelled protein elutes first (around 10-20 ml) and has high A280 and A450. The free label elutes much later in a broad peak and has much higher A450 than A480.

FIG. 3: A graphical representation of the DMI-derived proteomic profile of Individual A, based on data taken from Table 2. The height of the bar from the origin represents the percentage of the population variance exhibited by this individual. The depth of colour represents the absolute, deviation of the signal from 1 arbitrary unit. Large, deep coloured boxes contain the majority of diagnostic information about the individual.

FIG. 4: Impact of iterative rounds of positive selection (at low protein density on the selection surface) followed by negative selection (at high protein density on the selection surface) on the bias of a phage library. Bias was calculated by direct ELISA for phage binding to serum albumin (A) or Fibrinogen (B) or PAI-1 (C) or TGF-β (D) according to the formula (A+B)/(C+D), expressing the direct ELISA result as fraction in the range 0 to 1 representing the total phage concentration required to obtain a half-maximal signal. Error bars are SEDs calculated by assuming A and B to be estimates of the same parameter and C and D to be estimates of the same parameter. Pour rounds of this selection protocol reduced the bias factor of this library by approximately 8 fold.

FIG. 5: A 256-point immunomic profile from a typical healthy individual is shown in the upper left panel. Most of the antibodies in this sample react with antigens at the very left hand side of the profile (sub-libraries 1-8). By contrast, the 256-point immunomic profile from a typical person with heart disease (lower left panel) shows reactivity with many more sub-libraries, right across the profile. Pattern recognition analysis (PLS-DA; right hand panel, circles=diseased, squares=healthy) confirms that these differences are completely diagnostic for the presence of heart disease, since the two groups are entirely separated in the first principle component.

Definitions

“(Library) component”: A single antibody, protein or other antigen, or a mixture of antibodies, proteins or antigens, that are attached to a uniquely coded pool of tags. There may be many individual tags composing such a component, but they will all have the same code. Similarly, there may be many molecules of the antibody, protein or antigen but they will be identical, or else all come from the same mixture.

“Library”: A plurality of individual components as described above. Each component within a library may comprise a different tag, thus allowing the components within the library to be distinguished.

“Master Library”: A library of components which is much larger and more complex than a DMI library. A DMI library can be generated by sub-selecting just a fraction of the components from a master library. Typically such a master library will be composed of more than 10 million components.

“DMI Library”: A library made up of components which is suitable for DMI. Typically, such a library will be composed of between 10 and 1 million components, more typically between 100 and 10,000 components.

“Tag”: Any method of rapidly and easily determining the identity of an antibody, protein or other antigen bearing the tag. Tags are distinguished from “Labels” (see below) by their categorical property: that is, tags need only contain nominal information (tag 1, tag 2, tag 3 and so forth) and not necessarily any continuous information (a variable ranging from 0 to infinity).

“Label”: Any method of rapidly and easily determining the amount of an antibody, protein or other antigen bearing the label. Labels are distinguished from “Tags” (see above) by their quantitative property: that is, labels need only contain continuous information (a variable ranging from 0 to infinity) and not necessarily any nominal information (label 1, label 2, label 3 and so forth).

“Specific Binding”: An antibody specifically binds to a protein or antigen when it binds with high affinity to the protein or antigen for which it is specific but does not bind, or binds only with low affinity, to other proteins. For example, the antibody may bind to the protein or antigen with 5 times, 10, 20 times, more affinity than to a randomly generated polypeptide or other molecule.

### DETAILED DESCRIPTION OF THE INVENTION

The method of the invention is generally termed “Differential Megaplex Immunoassay” technology (DMI) herein. This strategy provides a relative abundance for each protein component in the proteome, compared to a reference sample (hence the term “differential”). It allows the analysis of thousands or even millions of proteins simultaneously (hence the term “megaplex”, which is a higher order extension of the conventional term multiplex). The key analytic technique exploited is the competition immunoassay (hence the term “immunoassay”).

1. DMI for Proteomic Profiling

In general terms, to perform a DMI experiment for proteomic profiling you require: an antibody library, a method of tagging the antibodies so that they can be uniquely identified, a reference sample, a method of labelling the reference sample and a strategy for reading the amount of label bound to each tagged antibody. Any or all of the components of the DMI experiment may be already known in the public domain, but the principle of combining these techniques in order to perform proteomic analysis is novel, and represents the invention described herein.

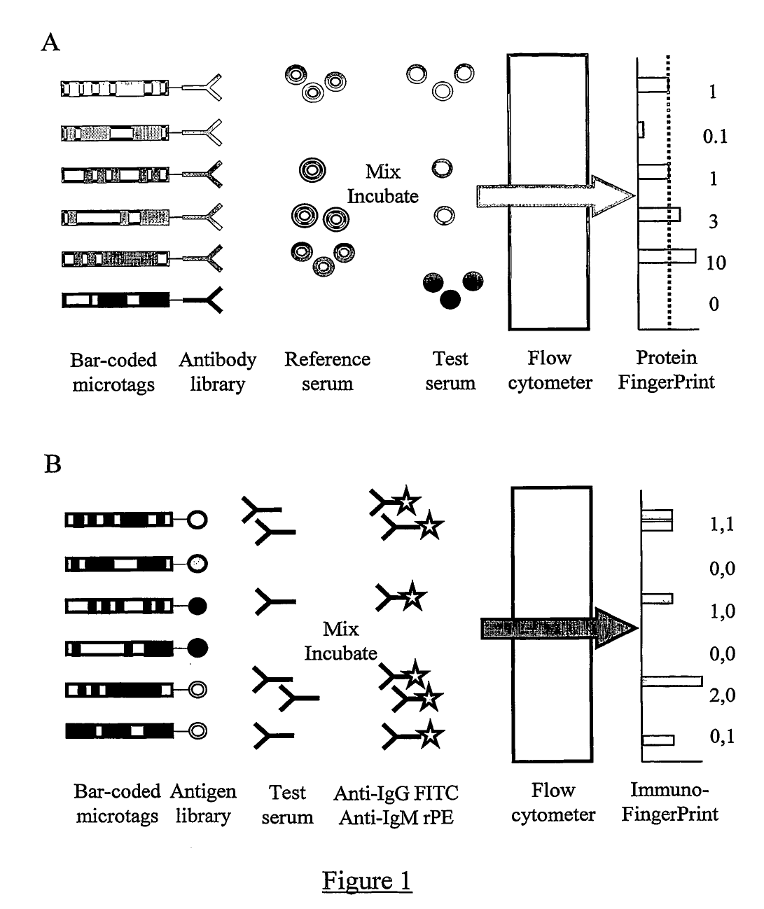

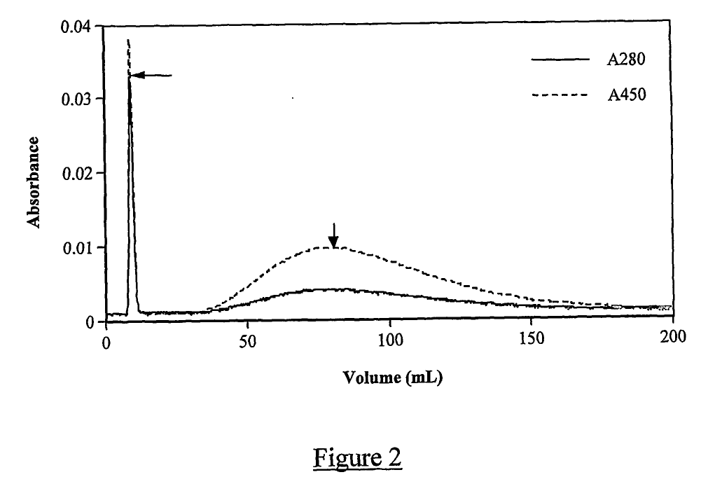

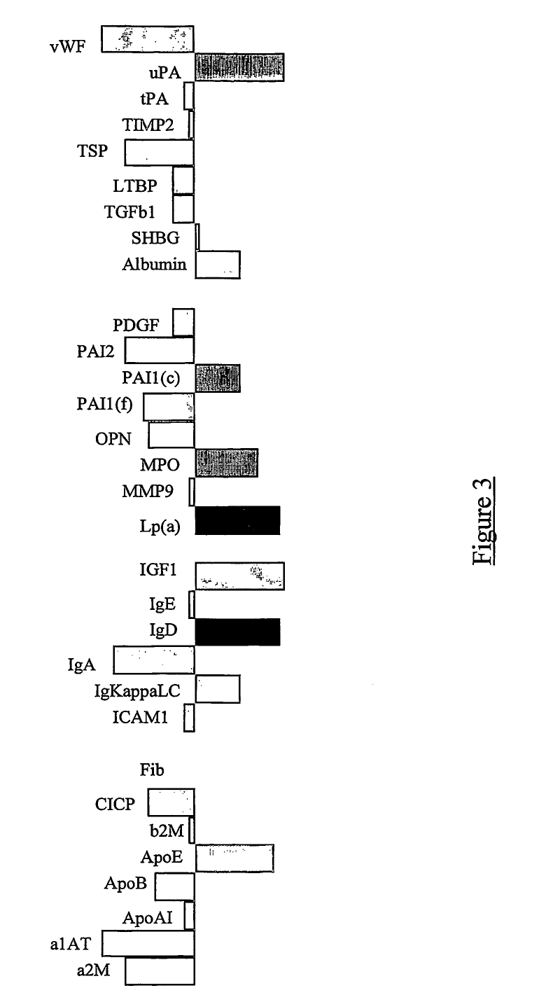

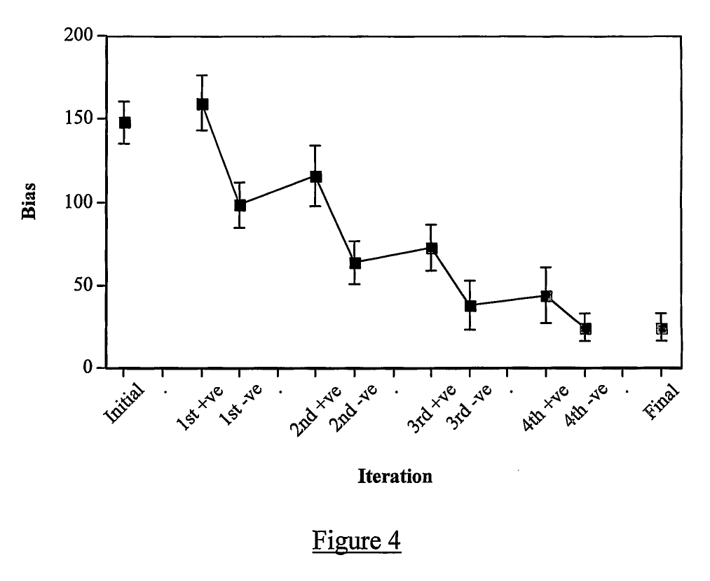

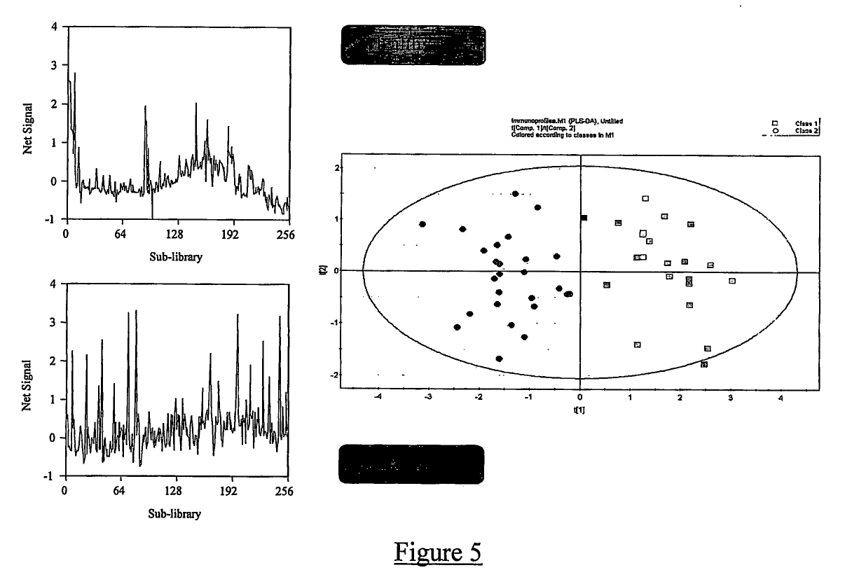

The general principle of the DMI experiment is as follows (see FIG. 1A):
1. Mix the labelled DMI reference sample with the sample under test, preferably in equal proportions;
2. Add the tagged antibody library and incubate together;
3. Read the amount of label bound to each tagged antibody.

First, the requirements for each of the key components of the experiment are described, followed by an exemplification of the general DMI experiment laid out above.

A: The Antibody Library

To be useful for DMI, the antibody library to be utilised should contain a significant number of antibodies which have as their cognate epitopes proteins that are present in the sample to be analysed. For example, to perform a proteomic screen using DMI on a human serum sample would require a library of antibodies a significant proportion of which recognised proteins present in human serum samples.

Ideally, such a library will also have a high degree of complexity: that is, that most, if not all, of the individual antibody species that compose the library, should recognise different proteins. In one embodiment, therefore, each of the plurality of antibodies used in the methods of the invention recognises and binds a different protein. Each antibody may recognise and specifically bind a different protein. Libraries with a high degree of redundancy, by contrast (where many of the antibody components recognise the same protein), will reduce the power of the DMI approach.

Ideally, the library should contain a large number of antibodies. An antibody library useful for DMI may contain between ten and 100 million antibodies, more typically between one hundred and 1 million antibodies.

The library must exist in a format where by the antibodies against different proteins are physically separated, or capable of physical separation. This ensures that each individual antibody component of the library can be uniquely tagged.

Antibody libraries with these properties can be constructed in a number of ways. For example, antibodies known to recognise components of the proteome of the sample to be investigated could be purchased individually from commercial antibody sellers, or else manufactured individually by the standard methods well known in the art. Libraries compiled in such a way are likely to be at the lower end of the size useful for DMI (typically 100 or less antibodies).

Alternatively, the library may be generated by phage display technology. A sample typical of those to be subsequently analysed by DMI may be coated onto a surface and used to positively select antibodies from very large general purpose libraries (such as those owned and generated by Cambridge Antibody Technology Limited, and similar companies). An antibody library generated in this way may, however, not comply with the ideal characteristics of a DMI antibody library in several ways—the redundancy may be relatively high and the population may be biased by the amount of each protein present in the positive selection mixture.

The present invention therefore provides a modification to the procedure well known in the art for selecting from phage display libraries which allow a low redundancy library with relatively little bias on amount of antigen present to be developed:

In order to reduce the bias of the library towards abundant species in the selection mixture, rounds of positive and negative selection are repeated iteratively, adjusting the total protein concentration applied to the selection surface. In the first round of positive selection, the selection mixture is applied at very low total protein concentration, for example from 0.1 μg to 100 μg per cm², to a very large surface area. This ensures that every protein the sample is efficiently represented on the surface. Phage are positively selected, released and grown up back up in number. This selected population is then subjected to a round of negative selection, where the same selection mixture as used in the first round is now applied to the surface at very high total protein concentration, for example 1 mg per cm² upwards, over a very small surface area. As a result, many of the phage directed against the abundant antigens bind to the surface and are lost from the population, whereas stochastically the rare proteins will hardly be represented on the negative selection surface where surface area for protein binding was limiting. The population of phage in the supernatant after negative selection are again grown up, and the process can be repeated iteratively with alternate round of positive selection and negative selection.

Preferably the high protein density selection is carried out at a protein density between 10 and 10,000 fold higher than the low protein density selection, more preferably between 100 and 1,000 times higher density. These ranges are based on the use of commercially available high-protein capacity plastic surfaces currently available (such as Nunclon plastics used to make ELISA plate wells) but may need to be adjusted accordingly for other substrates with different total protein binding capacities. Typically, the low protein density selection should be performed between 100 and 1-fold lower density than the nominal protein binding capacity of the substrate, preferably about 10-fold lower. The high protein density selection should be performed between 1-fold and 100-fold higher density than the nominal protein binding capacity of the substrate, preferably about 10-fold higher. The higher the high protein density coating concentration is relative to the nominal protein binding capacity of the substrate, the more extreme will be the change in library bias.

The bias of the library may be assessed as follows: the number of individual library components which bind to two different proteome components which are known to be highly abundant in the samples of interest (in the case of serum, these might be albumin and fibrinogen, for example) are determined. Similarly, the number of library components binding to two rate proteome components are also determined (cytokines such as TGF-beta and MCP-1 would be suitable markers for human serum). Direct ELISA may be used to quantitate the fraction of the total library elements that bind to each of these four marker proteins. The bias of the library would be calculated as (A+B)/(C+D) where A and B are the number of library elements binding to the abundant protein markers, and C and D are the number of library elements binding to the rate protein markers. Initially, after the first round of positive selection, this Bias Factor may be 1,000 or more. After several iterative rounds, the Bias Factor will approach 1.

The Bias Factor of the resulting library may decline faster if the ratio of the protein density on the selection surface during positive selection to the protein density on the selection surface during negative selection is stepwise reduced as the number of selection rounds is iterated. An example of such a selection protocol is illustrated in FIG. 4.

A DMI Antibody Library generated by phage display approaches will likely contain 10,000 to 10 million distinct antibody components and will, therefore, likely be at the upper end of library size useful for DMI.

To allow for unique tagging of each antibody component, the DMI antibody library may need to be formatted in a manner that physically separates the library components. For libraries where each component is generated individually, the components could be dispensed one at a time into multiwell plates, for example, at a known antibody concentration. For libraries generated by phage display approaches, multiple individual phage clones could be grown up, for example in multiwell plates, and the antibody concentration normalised in each well.

B: Method for Tagging the Antibody Library

DMI requires that each antibody component of the library be uniquely tagged in a manner that allows the antibody to be identified when in a mixture. Any method of tagging which allows the antibody to be identified, while still retaining its ability to specifically bind to its antigen, would be suitable for use in DMI.

Examples of suitable tagging methodologies would include:

Aluminium bar codes (such as those developed by Sentec Ltd). These bar codes are 100 μm×10 μm×1 μm aluminium strips which have holes punched in them, allowing millions of unique codes to be stamped onto them. They are produced using semiconductor chip fabrication methodology to very high specification. Each tag code is handled separately, for example in different wells of multiwell plates. The tag and the antibody can be coupled together by any method obvious to those skilled in the art, including heterobifunctional crosslinking or by charge-coatings applied to the tag. Any method that irreversibly couples the tag to the antibody without denaturing the antibody would suffice.

Dye-impregnated beads (such as those developed by Luminex). The beads have dyes with unique spectral properties impregnated into them, which can be used to unambiguously identify the bead. Dye-bead technology would likely only be useful for smaller DMI antibody libraries (less than approximately 100 antibody components) because of the limited availability of enough different suitable dyes. The bead and the antibody could be coupled together by any method obvious to those skilled in the art, including heterobifunctional crosslinking or by charge coatings applied to the bead.

Each tag may be linked to one or more antibody species. In one embodiment, each antibody species within the library is linked to a different tag so that the binding of each antibody may be assessed separately. Alternatively, two or more antibody species may be linked to a tag. For example, different antibody species which bind the same or different epitopes in a target protein may be pooled and linked to a single tag. In this way, all antibody binding to that target protein may be determined by assessing the label associated with that tag.

Irrespective of the tagging technology used, the ratio of antibodies per tag could be controlled, depending on the coupling chemistry selected. For DMI applications it would be desirable to have a large number of antibody molecules attached to one tag (from 10¹¹ to 10¹⁵ or more antibody molecules per tag) since the signal to noise ratio for reading the bound label will increase with increasing antibody density on the tag.

C: The Reference Sample

DMI is a differential assay methodology: it does not measure the absolute level of any analyte within the test sample, but estimates the ratio of the amount of the analyte in the test sample compared to a reference. Consequently, each DMI experiment requires a reference sample. The reference sample should be the same is for every DMI experiment where the resulting protein profile data are to be compared.

The reference sample should be of similar overall composition to the test samples—it should contain the same analytes in approximately the same concentrations as the test sample. For example, a reference sample may be obtained from the same tissue as the test samples. A reference sample may be obtained from the same species as the test samples. Preferably, the reference sample is obtained from the same tissue in the same species as the test samples. DMI shows excellent quantitative resolution where the ratio of the analyte is close to 1 (say, in the range 0.1 to 10) but outside these ranges the signal gradient declines sharply. Consequently, to obtain the highest data density in the resulting protein profile, the concentration of each analyte in the reference sample would ideally be equal to the average of the analyte concentration in all the test samples.

One method of generating such a reference sample would be to take a small amount of all the samples to be tested and pool them, mixing thoroughly. The resulting pool would have the ideal properties of a reference sample for DMI.

Another method for generating a reference sample would be to make a pool of samples of similar origin to the test samples, but not actually including the test samples. The use of pooled reference samples increases the likelihood that: (a) every analyte present in the test sample will be represented in the reference sample and (b) that the concentration of each analyte in the reference sample approaches the average value for all the test samples.

As an example, to create a reference sample for a DMI experiment examining human serum samples; aliquots of serum from many different human subjects may be taken and pooled. To create a reference sample for a DMI experiment examining cultured liver cells, protein extracts from many different cultures of liver cells would be taken and pooled. It would not be appropriate to use a pool of human liver cell extracts as the reference sample for a DMI experiment examining human serum samples.

After labelling (see below), the reference sample should be at approximately the same total protein concentration as the average of the test samples. If necessary, the total protein concentration of the labelled reference sample should be adjusted prior to beginning the DM experiment.

D: A Method for Labelling the Reference Sample

The reference sample is labelled such that a plurality of proteins within the sample bear the label. In a preferred embodiment, the reference sample is labelled in such a fashion that all of the protein components within the sample are labelled to some extent. Each different protein component may or may not labelled to the same extent as all the others.

Any label may be used which can be read easily and rapidly once bound to the tagged antibodies. For example, the label may be a fluorescent dye that can be read by interrogating the tagged antibody with a laser, inducing fluorescence, which can be quantitated with a photodetector.

Suitable fluorescent dyes include: fluorescein, oregon green, GFP, rhodamine, r-Phycoerythrin, Cy3, Cy5, coumarin, AMCA, texas red, Alexa Fluor dye series (350, 430, 488, 532. 546, 555, 568, 594 and 633) and BODIPY series (493/503, FL, R6G, 530/550, TMR, 558/568, 564/570, 576/589, 581/591, TR, 630/650-X and 650-655-X). Providing appropriate post-processing steps are utilised (which are well known in the art) then lanthanide chelates can be used as labels (for example Europium chelates) which are read using laser-induced fluoresence which has a very long lifetime, allowing time-resolved fluorescence reading to improve signal to noise ratios. Alternatively, a non-fluorescent label could used. Suitable non-fluorescent labels include: radioactive decay (for example: tritium, iodine-125, phosphorus-32, sulphur-35 labels; read using a suitable scintillation counter), gold particles of various sizes (read using a microscope, preferably with automated image analysis software to identify and count the particles) and chemiluminescent probes (for example luciferase label read by exposing it to luminol-containing buffer in a luminometer).

The chemistry used to couple the label to the protein components of the reference sample must meet three criteria: (a) it must irreversibly couple the label to the protein (b) the protein must not be denatured by the process and (c) the label must still be detectable after the coupling reaction. Any chemistry that meets these criteria can be used. For example, fluorescein isothiocyanate can be reacted with the protein fraction of the reference sample. After removal of unconjugated fluorescein e.g. by column chromatography) the labelled sample can be reconstituted to a total protein concentration equal to the approximate average of the test samples.

The labelling ratio (the number of labels per protein molecule) can vary within a reasonable range for a DMI reference sample. Typically it will be in the range 0.1 to 50 labels per protein, more typically in the range 1 to 5. Low labelling ratios reduce the sensitivity of the detection system, and increase noise, while high labelling ratios can affect the ability of the labelled protein to bind to its cognate antibody in the tagged antibody library.

E: Strategy for Reading the Amount of Label Bound to Each Tag

The strategy for reading the amount of label bound to each tag will depend on the nature of the tag and the label. In order to generate data-rich protein profiles the reading method should be relatively high throughput. However, for small DMI antibody libraries (e.g. less than a few hundred antibody components) the label could be read manually. For example, using a microscope each tagged antibody in turn could be identified and the tag read, then the amount of label determined. Reading the tag might involve, for example, taking a spectrum of the tagging dye or reading the aluminium bar code under transmission illumination. Reading the label might involve, for example, counting bound gold particles or capturing induced fluorescence with a photomultiplier.

For larger DMI antibody libraries (with thousands or millions of antibody components) an automated strategy for reading each tagged antibody component will be required. For example, the tagged antibody components could be passed one at a time through a standard flow cytometer. In the example where the tag is an aluminium bar code and the label is a fluorescent dye, the flow cytometer (with appropriate software) could read both the tag and the bound label.

Successful DMI requires that both the reading of the tag and the bound label be performed with high fidelity and reproducibility. For example, for the determination of bound label on a bar-code tagged antibody, a standard flow cytometer can read the tag correctly with an error rate of less than 1 in 10,000, while the estimate of bound fluorescent label can be performed with a repeated measures coefficient of variation below 5%. With these characteristics, DMI approaches the robustness of methods such as NMR-based metabonomics, while retaining the ease, speed and cost benefits of gene array technology.

F: The Procedure

The labelled reference sample, adjusted to the same total protein concentration as the average of the test samples, is then dispensed at an appropriate volume into tubes or microtitre plate wells. Typically volumes between 1 μl and 200 μl will be used.

Next, each test sample is added one well at a time. The volume of test sample is preferably equal to that of the labelled reference sample. The plate must then be mixed thoroughly, to ensure the test and reference samples are homogeneously distributed.

An appropriate volume of the mixed antibody library must then be added. Typically between 1 μl and 100 μl of library will be added. The number of individual tags to be added will depend on the complexity of the library, as well as its redundancy and bias factors. Typically, between 10 and 200 times more individual tags will be added than there are non-redundant components of the library. After addition of the library, the reaction tubes or plates must be mixed thoroughly, and incubated under conditions suitable for the binding of the antibodies to their targets, for example for a period to allow the antigens in the test and reference samples to bind to their cognate tagged antibodies. Typically, this will be for a period between 10 and 180 minutes. Typically, the reactions will be continually agitated throughout the incubation to ensure that the tags remain randomly suspended within the liquid. Typically, the incubation will be performed between 4° C. and 37° C. Other components may be added to the reaction as appropriate, to improve the specificity and selectivity of antibody binding to antigen: typically, a non-ionic detergent is added at a concentration between 0% and 1% volume/volume (for example, Tween 20 at 0.1% v/v). Similarly, the salt concentration can be varied: typically, sodium chloride solution is added to increase the total salt concentration by between 0 mM and 250 mM. Similarly, the divalent cation concentration can be varied: typically, calcium chloride or magnesium chloride are added to increase the calcium or magnesium ion concentration by between 0 mM and 10 mM as required, or EGTA is added to decrease the calcium and magnesium concentrations as required. Similarly, the pH of the reaction can be varied: typically, 1M hydrochloric acid or 1M sodium hydroxide are added to reduce or increase, respectively, the pH of the reaction by between 0 and 3 units.

At the end of the reaction, the interaction between antigen and antibody is typically terminated. Several methods can be used: for example, the reactions can be diluted substantially (typically by 5 to 50 fold with buffered saline); alternatively, the reaction can be rapidly cooled (typically to 4° C.); alternatively a crosslinking reagent can be added (typically formalin is added to a 3% final concentration).

Following termination of the reaction, the tagged antibodies can be directly read or they can be washed by gentle ultrafiltration and then resuspended at an appropriate concentration prior to reading. Whether the tagged antibodies need to be washed prior to tagging will depend on the method of reading. Typically, using a fluorescence microscope or a flow cytometer, no washing step is necessary.

The amount of label bound to each tag must then be determined. The number of tags which must be read varies depending on the complexity of the library, as well as its redundancy and bias. Typically, between 2 and 200 tags will be read for each non-redundant component of the library. The smaller the library, the larger the number of tags per component that can be read. If low numbers of tags per component are read for very large libraries, then a significant number of components in the final vector will have to be recorded as data missing values. Where more than one tag representing the same component is read, the amount of label bound to each is typically averaged before reporting the final vector.

The resulting output vector can then be analysed in a number of ways. Typically, a number of vectors from different individuals are used to construct the X-matrix for various megavariate statistical analyses, including PCA, PLS-DA and OSC. Such methods allow the individuals to be classified according to some pre-existing phenotype (such as disease status). Once a model has been constructed classifying individuals whose phenotypic status is known the model can then be used to predict the phenotype of individuals whose status is unknown. This is the basis of the application of DMI proteomic profiling to medical diagnostics.

The DMI approach has a number of advantages over current proteomics platforms. In particular, existing methods can be limited in sensitivity to the relatively abundant components in the mixture. For example, when applied to serum, the very high levels of albumin in the sample can hamper traditional approaches. However, provided that the antibody against albumin is present only once in the tagged DMI library then albumin will contribute only one date point to be protein profile. DMI is also quantitatively robust, with coefficients of variation below 5% for most antibodies, and therefore substantially superior to microarray-based proteomic platforms.

2. DMI for Immunomics

One major gap in the “coverage” of a genomic, proteomic and metabonomic profile is the organisation of the mammalian immune system, at least if conventional proteomic approaches are used. For example, antibodies (one of the important effector arms of the adaptive immune system) are not efficiently resolved on the basis of their antigen specificity in any conventional multi-omics profile. All antibodies of a particular heavy chain class appear overlaid as a single protein in conventional proteomic profile, masking the tremendous variation in antigen specificity between different antibody clones.

Immunomics is a newly coined term for a highly specialised example of proteomics: analysis of the population of antibody molecules produced by a given individual at a given time. This information is not normally encoded within a proteomic profile (whether generated by DMI or classical methods). It is also absent from genomic, transcriptomic or metabonomic datasets. Consequently, specialised techniques will be required to perform high throughput analysis of the immunomic repertoire. To date, there are no publicly disclosed methods for performing immunomics. Consequently, a second important application of the DMI principle is as a first high throughput, robust and reproducible method for obtaining an immunomic dataset.

The present invention addresses this issue, by designing and implementing strategies to profile the entire portfolio of antibodies in a biological specimen, such as serum. This profile is termed an “immunomic” profile, because it provides an overview of the current status of the immune system in a given individual. In principle, it is possible to envisage implementations of immunomics which look at other aspects of the immune system as well: there are methods already established for examining antigen-specific T cell clones, although to date there no attempt to profile the entire T cell repertoire of an individual has been published. Such an immune cell profile would also be an implementation of immunomics.

In general terms, to perform a DMI experiment for immunomics you require: an antigen library, a method of tagging the antigens so that they can be uniquely identified, one or more labelled anti-immunoglobulin antibodies and a strategy for reading the amount of label bound to each tagged antibody. Any or all of the components of the DMI experiment may be already known in the public domain, but the principle of combining these techniques in order to perform immunomic analysis is novel, and represents the invention described herein.

The general principle of the DMI experiment is as follows:
1. Mix the tagged antigen library with a test sample;
2. Detect bound antibody with a panel of labelled anti-immunoglobulin antibodies;
3. Read the amount of label bound to each tagged antibody.

First, the requirements for each of the key components of the experiment are described, followed by an exemplification of the general DMI experiment laid out above.

A: The Antigen Library

The requirements for the antigen library for immunomics are very similar to the requirements for the antibody library for proteomic profiling: the library should be as large as possible with low redundancy (preferably with any given antigen only represented by a single component of the library).

A suitable antigen library may comprise oligopeptides and/or oligosaccharides. The source of the antigens can either be by manual assembly of the library using purified protein and non-protein antigens as individual library components (analogous to the manual assembly of an antibody library using purified antibodies) or generated by combinatorial chemistry. For example, a peptide antigen library could be generated by standard solid phase chemistry, using methods well known in the art.

As with the antibody library, the components of the antigen library must be capable of being separated (or else be generated separately) so that they can be dispensed individually (for example, into microtitre plates) to allow them to be tagged.

One approach to obtain a crude immunomic profile is based on the generation of an antigen library which is then exposed to the antibody-containing sample (usually serum) and the amount of antibody binding to each library elements then being determined. The problem with this approach is there are essentially an infinite number of possible antigens, so some criteria must be adopted to limit the size of the library,

One solution is to limit the library to peptide antigens, because of the ease with which peptide libraries can be synthesised by combinatorial chemistry strategies. Using a library of peptide antigens in this way limits the resulting profile to those antibodies which recognise a simple linear antigen (and specifically excludes structural epitopes with contributions from discrete parts of a larger polypeptide chain). Nevertheless, antibodies against simple linear peptide antigens are known to be common in polyclonal sera, although the fraction of the total pool of antibody clones in a typical individuals which fall into this class has not been estimated.

Any length of peptide sequence could be used in an antigen library. For example peptides of 4, 5, 6, 7, 8, 9, 10, 12, 15, 18, 20 or more amino acids in length may be used. However, the shortest peptide sequence which is robustly recognised by anti-peptide antisera is about 8 amino acids in length. A preferred library will therefore consist of peptides of at least 8 amino acids in length, for example 8 or more, 10 or more, 15 or more, 20 or more 30 or more, 40 or more or 50 or more amino acids in length.

A library of all possible octapeptide sequences would have 20⁸ (or approximately 25 billion elements), and could not be practically handled. The two options to reduce the library size would be to reduce it complexity (so that it is no longer comprehensive) by selecting a subset of all the possible library elements, or to pool the library elements to generate a manageable number of sub-libraries thereby retaining the comprehensive nature of the library but reducing the resolving power of the resulting profile.

For pooling methods, any number of pools may be used. The number of pools chosen will depend on the overall number of library elements, the number of sub libraries required and the number of elements per sub library required. For example, in a library of all possible octapeptide sequences as described above, 262,000 sub-libraries each containing almost 2 million sequences could be generated. A simplified library might contain 512 sub-libraries of around 50 million sequences. Alternatively a simpler library of 256 octapeptide sub-libraries, with approximately 100 million different sequences each can be generated.

By dividing a large library into sub-libraries in this way, the methods of the invention may be carried out wherein rather than each individual library member being tagged, each group or sub-library of library members received a different tag. This will not enable a direct assessment of the specific library member that is bound during the assay, but can dramatically reduce the number of individual tags required. It is still possible to obtain a useful immunomic profile using a library comprising individually tagged groups or mixtures of library members, for example peptides.

The individual members of a library may be sub-divided into groups by any criteria or randomly. For example, in the case of a library of peptides, the sub-libraries may comprise a mixture of peptides which are selected on the basis of their amino acid sequence. It may thus be possible to use such a library to obtain some basic amino acid sequence information about the peptides being bound in the assay, even though the specific sequences being bound cannot be determined directly. It is, of course, possible to further refine the results of such an assay by taking the components of the particular mixtures or sub-libraries of interest and further assaying them, for example by dividing them into smaller groups or by tagging each peptide individually.

Any suitable method can be used to produce a mixture of peptides or a library of mixtures suitable for use in the methods of the invention. For example, a suitable mixture may be a mixture of peptides wherein each peptide is of length n amino acids and of the formula:
X₁—X₂—X₃— . . . —Xₙ
wherein:
each X represents an amino acid independently selected from one of a number of groups of amino acids;
each group of amino acids consists of less than 20 different amino acids,
n is the same for all peptides present in the mixture;
all of the following amino acids are present in at least one group: arginine, lysine, histidine, glutamate, aspartate, proline, cysteine, serine, threonine, tryptophan, glycine, alanine, valine, leucine, isoleucine, methionine, asparagine, phenylalanine, tyrosine and glutamine, and
for each peptide in the mixture the amino acid at the same position is selected from the same group.

Using such a mixture, it is known for all peptides in the mixture which group of amino acids each amino acid position must be selected from. The mixture may therefore include a wide variety of individual peptides as variation may occur at all amino acid positions, but some sequence information will be available.

In such a mixture of peptides it is possible to specify that no amino acid is present in more than one of the groups of amino acids, i.e. that each amino acid will only appear when it's group is selected at a particular position. It is further possible to specify that each group of amino acids contains the same number of different amino acids. Thus for the twenty amino acids listed above, one could envisage dividing them into two groups of tem amino acids, four groups of five or five groups of four.

For example, the twenty amino acids could be subdivided by type as follows: GROUP 1 Arg, Lys, His, Asp, Glu (charged); GROUP 2 Gly, Ala, Leu, Ile, Val (small hydrophobic); GROUP 3 Met, Phe, Pro, Tyr, Trp (large hydrophobic) and GROUP 4 Ser, Thr, Asn, Gln, Cys (hydrophilic).

An alternative grouping is shown in Table 5 below, in which the amino acids are allocated to groups “I” and “B”. The “I” group contains the majority of the amino acids likely to have the most significant effect on antigenic structure and antibody binding affinity, and consequently this division of the amino acids into the two pools should maximise the specific binding of any given antibody to sequences within a single mixture or sub-library.

A library may thus be generated of such peptide mixtures. For example a library may be generated wherein all the peptide contained therein has the same amino acid length. A suitable library may be one in which no peptide is present in more than one library, i.e. all members of the library have been divided into groups for example on the basis of amino acid sequence. Where the library consists of a number of mixtures as described above, preferably each of the mixtures in the library will have been generated using the same groupings of amino acids, allowing a direct comparison of the mixtures on the basis of the amino acid groupings. Preferably the mixtures within the library will differ by virtue of the fact that the combination of groups chosen to obtain the peptides differs between the mixtures. The library may thus comprise mixtures representing all possible combination of the groups. For example where the 20 amino acids are divided into two groups of 10, at each amino acid position in the peptide, an amino acid from one or other group may be present. A library constructed in this way may thus contain a mixture of peptides representing each possible combination of the groups at each position. The library may thus contain 2ⁿ mixtures where n is the length of the peptide sequence. Thus, if the peptides were 8 amino acids long one might envisage using a library of 256 peptide mixtures based on a division of the amino acids into two groups. The library may thus comprise all possible peptides of length n, each being present in only one mixture.

The sub-libraries may be synthesised by any conventional method, for example by an adapted version of standard solid-phase peptide synthesis protocols by Affiniti Research Products Ltd. Most synthesis protocols do not give equal yields for all possible amino acid couplings. In particular, sequences with a high content of hydrophobic amino acids (which dominantly compose the “B” group) are likely to be synthesised in lower yield than the more hydrophilic sequences. Thus, it is likely that certain sequences are over- or under-represented in each sub-library to an extent which cannot readily be determined. However, it is important to note that the synthesis protocol is extremely tightly controlled, so that the same sub-libraries (with the same synthetic sequence biases) can be repeatedly synthesised even though the nature and extent of the bias within the individual sub-libraries is not known. Along similar lines, the different sequences which compose the sub-libraries will have different solubilities in aqueous buffers, and this may also result in biased representation of the different sequences within the sub-library. To minimise this, each sub-library can be dissolved in a solvent such as 100% DMSO. In the examples set out below, the sub-libraries were dissolved in 100% DMSO to yield a 10 mM stock solution which was subsequently diluted in aqueous buffers

Once the sub-libraries are designed and synthesised, various methods can be used to determine the amounts of antibody which bind to each pool of antigens. The most straightforward method is a solid phase immunoassay: each sub-library is coated onto an ELISA plate well, and is then exposed to a human serum sample. After washing, bound antibody is detected and quantitated using a labelled anti-human IgG detection antibody. Using any kind of solid phase immunoassay approach sets up a competition between antibodies of different classes (and indeed different clones) for each of the antigen sub-libraries. Consequently, it is possible to generate profiles in which each of the immunoglobulin sub-classes is detected separately. For example, an IgM detection antibody, an IgE detection antibody, an IgD detection antibody, an IgA detection antibody, a specific IgG detection antibody (e.g. an IgG1, IgG2a, IgG2b, IgG3 or IgG4 detection antibody) a pan-IgG detection antibody capable of detecting all IgG subtypes, or an antibody capable of detecting two, more or all of these antibody sub-classes and subtypes can be used. Depending on the detection antibody used, it is important to appreciate that low signal on a specific sub-library might indicate low prevalence of the particular sub-class or subtype of antibody for which the detection antibody is specific, or it might reflect very high prevalence of antibodies of a different sub-class. In this context, it is important to remember that the competition between antibodies for a surface-bound antigen will depend on a variety of factors, including relative prevalence, affinity and avidity of the competing antibody pools.

Once the library has been designed, any one of a large number of immunological methods can be used to obtain an immunomic profile. These can be broadly divided into two groups: “uniplex” methods where antibody binding to each library element is determined separately, and then combined to yield to the profile and “multiplex” methods where antibody binding to each library element is determined in the same tube, yielding the complete profile from a single reaction. Clearly, multiplex methods have the advantage of simplicity (indeed they are currently the only viable option if the number of library elements exceeds a couple of hundred) and they also require less sample, but they may also not be so simple to interpret: it is possible that the antibody capable of binding to a range of different library elements is actually the same antibody pool with relatively relaxed antigen specificity. In such cases, there will be competition for binding between the library elements in the multiplex method but not in a uniplex method. Such competition might amplify or minimise the differences between individuals, and only empirical study can determine whether multiplex or uniplex profiles will be the most useful for any given application.

A typical uniplex method would be a solid phase immunoassay. Individual library elements are coated onto high protein binding wells (such as Nunc Maxisorp), non-specific binding is then blocked before the each library element is exposed to the serum sample under analysis. Unbound antibody is washed away, and bound immunoglobulin detected using an appropriately labelled detection reagent (such as an animal anti-human IgG conjugate). After exposure to a chromogenic substrate, the absorbance from each library element (net of background absorbance from wells coated with buffer alone) is plotted to yield the immunomic profile.

A typical multiplex method utilises a tagging method to label each library element separately so that the binding of antibody to each library element can be assayed simultaneously in a single reaction. Examples of such tagging technology are the aluminium barcoded particles (termed UltraPlex particles) developed by SmartBead, or the dye-impregnated beads developed by Luminex described herein. In both cases, individually coded particles (uniquely identified either by the bar code or the spectral properties of the dye) are coated with a particular library sub-element, before being mixed together and exposed to the serum sample under analysis. After antibody binding, washing and detection steps identical to those used in the solid phase assay, the amount of antibody bound to each coded particle is determined separately. In practice, the amount of binding to a number of particles of each code is determined, and averaged, in order to construct a reliable profile.

B: A Method of Tagging the Antigen Library

All of the same considerations that applied when tagging the antibody library described above apply to tagging the antigen library, and the same methods are likely to be useful. Where the library components are proteinaceous, then the antigen library can be treated exactly as if it was an antibody library. Where the library is composed of oligopeptides, then consideration of the tagging can be incorporated into the synthetic chemistry used to generate the antigen: for example, a chemical linker can be added to every peptide during synthesis, and this linker can be used to attach the peptides to the tags. The precise nature of the linker would vary depending on the nature of the tag. For dye-containing latex beads, for example, a bifunctional succinamide crosslinker could be utilised. Where the library is composed of oligosacharrides, then the sugar chains can be attached to a carrier protein and then the library be treated as for a protein library, or else a suitable crosslinker can be added to the sugar chains during synthesis, as for the peptides.

C: A Panel of Anti-Immunoglobulins Appropriately Labelled

Whereas, for proteomic profiling the label is applied to the reference sample, and the amount of each protein in the test sample is measured indirectly by competition with the labelled reference sample, for immunomics the antibody that binds to each tagged antigen is directly detected. This requires a panel of anti-immunoglobulins, or equivalent reagents, which bind to immunoglobulins with high affinity and specificity.

The anti-immunoglobuline should be specific to the types of immunoglobulin likely to be present in he test sample. For example, the anti-immunoglobulins may be specific to immunoglobulins from the same species as the test sample, e.g. anti-human immunoglobulins where the sample is derived form a human.

Suitable immunoglobulin panels are readily available from commercial sources—for example, the WHO standard antibodies for detecting human immunoglobulins can be used. In the ideal experiment, a panel of one or more such antibodies would be used as detection reagents, one specific for each of the heavy chain classes of immunoglobulin found in the required species. For example, a panel of antibodies specific to one or more of the heavy chain subclasses in humans (IgG1, IgG2a, IgG2b, IgG3, IgG4, IgA, IgD, IgE and IgM) may be used. Suitable types of detection antibody are described above. The WHO standard antibodies are mouse monoclonal antibodies, and are consequently available in large, and essentially inexhaustible batches of detection reagents with identical properties.

The selected detection reagents must then be labelled using any method suitable for high throughput detection as described above in relation to the labelling of the reference sample in proteomics. For example, the WHO standard antibodies can be labelled with fluorescent dyes. A different dye may be used for each different detection reagent (for example, anti-human IgG1 could be labelled with fluorescein, while the anti-human IgM could be labelled with r-Phycoerythrin). There are plenty of spectrally distinguishable fluorescent dyes available to allow all nine of the WHO standard antibodies to be separately quantitated.

As for the labelling of the reference sample for protein profiling, the only other requirement for the label is that it does not affect the detection characteristics of the detection reagent once the label is applied, and that the label can still be read once it has been bound to the detection reagent. The same requirement applies here.

D: A Strategy for Reading Label Bound to the Tagged Antigen Library

All of the considerations that applied to reading a tagged antibody library for DMI proteomic profiling, also apply identically to reading a tagged antigen library for DMI immunomic profiling.

E: The Procedure

The test samples, e.g. serum samples are added one well at a time, dispensing an appropriate volume of each (typically 1 μl to 200 μl).

An appropriate volume of the mixed antigen library is then added. Typically between 1 μl and 100 μl of library will be added. The number of individual tags to be added will depend on the complexity of the library. Typically, between 10 and 200 times more individual tags will be added than there are components of the library. After addition of the library, the reaction tubes or plates must be mixed thoroughly, and incubated under conditions suitable for the binding of any antibodies present in the test sample to their targets, for example for a period to allow the antibodies in the test serum to bind to their cognate tagged antigens. Typically, this will be for a period between 10 and 180 minutes. Typically, the reactions will be continually agitated throughout the incubation to ensure that the tags remain randomly suspended within the liquid. Typically, the incubation will be performed between 4° C. and 37° C. Other components may be added to the reaction as appropriate, to improve the specificity and selectivity of antibody binding to antigen: typically, a non-ionic detergent is added at a concentration between 0% and 1% volume/volume (for example, Tween 20 at 0.1% v/v). Similarly, the salt concentration can be varied: typically, sodium chloride solution is added to increase the total salt concentration by between 0 mM and 250 mM. Similarly, the divalent cation concentration can be varied: typically, calcium chloride or magnesium chloride are added to increase the calcium or magnesium ion concentration by between 0 mM and 10 mM as required, or EGTA is added to decrease the calcium and magnesium concentrations as required. Similarly, the pH of the reaction can be varied: typically, 1M hydrochloric acid or 1M sodium hydroxide are added to reduce or increase, respectively, the pH of the reaction by between 0 and 3 units.

At the end of the reaction, the tags are washed by gentle ultrafiltration, typically with phosphate buffered saline. Other components, such as non-ionic detergent can be added to the wash buffer to improve the specificity and selectivity of antibody binding to antigen. Typically, Tween 20 is added at 0% to 1% volume/volume final concentration.

After washing, the tags are resuspended in a buffer containing the panel of labelled detection reagents. For example, where the test sample is from a human source, anti-human immunoglobulin antibodies are used as detection reagents at a concentration between 0.05 and 50 μg/ml for each individual antibody (more typically between 0.5 and 5 μg/ml). Additional components can be added to the incubation buffer to improve the specificity of detection reagent binding to the captured antibody on the tags. These are the same components that could be added during the initial reaction of the library with the test samples. The labelled detection reagents are then typically incubated with the tagged library for between 10 and 180 minutes. The reactions are typically agitated for the period of the incubation to keep the tags randomly suspended in the liquid. The incubation is typically performed at between 4° C. and 37° C.

At the end of the reaction, the tags may be washed by gentle ultrafiltration, typically with phosphate-buffered saline. Other components, such as non-ionic detergent can be added to the wash buffer to improve the specificity and selectivity of antibody binding to antigen. Typically, Tween 20 is added at 0% to 1% volume/volume final concentration. Whether the tagged antibodies need to be washed prior to tagging will depend on the method of reading. Typically, using a fluorescence microscope or a flow cytometer, no washing step is necessary.

The amount of label bound to each tag must then be determined. The number of tags which must be read varies depending on the complexity of the library, as well as its redundancy and bias. Typically, between 2 and 200 tags will be read for each non-redundant component of the library. The smaller the library, the larger the number of tags per component that can be read. For each tag, the amount of each different label (representing each of the different heavy-chain classes of immunoglobulin) will be read separately. Depending on how many immunoglobulin classes were separately detected, the output vector will have between one and nine times more values than there are non-redundant components to the library. If low numbers of tags per component are read for very large libraries, then a significant number of components in the final vector will have to be recorded as data missing values. Where more than one tag representing the same component is read, the amount of label bound to each is typically averaged before reporting the final vector.

The resulting output vector can then be analysed in a number of ways. Typically, a number of vectors from different individuals are used to construct the X-matrix for various megavariate statistical analyses, including PCA, PLS-DA and OSC. Such methods allow the individuals to be classified according to some pre-existing phenotype (such as disease status). Once a model has been constructed classifying individuals whose phenotypic status is known, the model can then be used to predict the phenotype of individuals whose status is unknown. This is the basis of the application of DMI proteomic profiling to medical diagnostics.

F: Interpreting the Profile

The amount of immunoglobulin binding to each of the sub-libraries will vary depending on the sequence composition of the sub-library elements. The variation in signal between control wells in the above assays which were coated with buffer alone allow the application of confidence limits for signal variation due to sub-library composition. Many sub-library elements will show antibody binding which is in the range expected for uncoated wells, suggesting that any antibody binding to the sequences within that sub-library is below the detection sensitivity of the assay. However, it is likely that some wells will show significantly less signal than the uncoated wells: the most likely interpretation for this is that very high levels of immunoglobulin of a different sub-class to that being detected is present and binding to the coated sub-library further blocking non-specific immunoglobulin binding. Where, for example, IgG is being detected, it is most plausible that any blocking antibodies are of the IgM sub-class whose pentameric structure gives high avidity for solid-phase binding. For other wells there may be significantly more signal than in the uncoated wells, suggesting specific immunoglobulin binding to at least a fraction of the related sequences composing the sub-library.

Ultimately, it is next desirable to identify the particular sequences responsible for the signal in sub-libraries that turn out to be of particular interest (perhaps because their signal is diagnostic for the presence of a particular disease). Further libraries with lower degeneracy could be synthesised where all the library elements have the same pattern of, for example, “I”-group and “B”-group amino acids as the single sub-library of interest from the master library. Alternatively, the e.g. 100 million sequences in the sub-library could be trivially fractionated on the basis of physical properties such as charge by chromatography. Both approaches, if used iteratively could eventually identify the particular sequences responsible for a given signal in the original broad immunomic profile.

A further approach that could be taken would be to establish the specificity of antibody reactivity with the sub-library sequences by determining the immunomic profile of a monoclonal antibody directed against a known octapeptide sequence.

Ultimately, however, the major tool for interpreting immunomic profiles such as those shown here will be to apply pattern recognition tools in an attempt to link particular signatures within the profile to phenotypes of interest.

One suitable pattern recognition tool is Principal Component Analysis (PCA). PCA is a megavariate statistical method ideally suited to the recognition of class-specific signatures in datasets with many more measured parameters (k) than observations (n). PCA is an unsupervised pattern recognition method (which means that the model derived is generated without knowledge of the disease status of any of the individuals) and is consequently robust to overfitting, and does not require external validation. It is possible to apply a supervised pattern recognition method (such as Partial Least Squares Discriminant Analysis, PLS-DA) which also yields excellent separation between the groups. However, such models do require external validation, whereby profiles not used to generate the model are queried against the model. If the model is robust it correctly predicts these external validation profiles, while if the model is over-fitted the external prediction is substantially less good than the internal predictivity.

A range of other pattern recognition methods known in the art could be applied to the methods of the invention, including, but not limited to: genetic computing, support vector machines, linear discriminant analysis, variable selection algorithms and wavelet decomposition. In addition, a range of pre-processing filters known in the art could be applied to the data prior to application of the pattern recognition algorithm, including but not limited to: orthogonal signal correction, binning, adaptive binning, scaling and fourier transformation. In each case, it is necessary to determine by empirical application of the various available techniques, either together or in combination, which method yields the best separation between the immunomic profiles of the diseased and healthy individuals.

The pattern recognition tools described herein may be used to predict the disease status of individuals who have not yet been medically diagnosed for a particular condition. The immunomic profile of the individual is obtained by the methods described herein, and that profile is used compared to the model derived as described herein. Depending on the position of the new profile, it is possible to make a prediction of the disease status of the individual. Any of a number of methods well known in the art can be used to make such a prediction, such as a Cooman's Plot.

The utility of the immunomics profile for diagnostic purposes will depend on a number factors: most importantly, there should be a stable element to the profile for a given individual on a time-scale similar to that over which the particular disease develops, and there should be differences between individuals in this stable element of the profile. If this is the case, then it is possible that signatures can be found which are diagnostic for the presence of certain diseases.

G: Strategies for Improved Immunomic Profiling

The basic methods described above may be modified in a number of ways. For example, the number and size of the sub-libraries can be varied.

A simple variation on the technique would be to measure the binding of different immunoglobulin sub-classes to the same sub-libraries. This might be possible by using detection reagents tagged with distinguishable labels: in the multiplex approach, detection antisera against different human immunoglobulin sub-classes could be tagged with different fluorescent labels allowing the amount of IgM, IgG1, IgG2 and IgD (for example) bound to each sub-library to be determined in the same reaction. Implementation of such a method would increase the data density of the basic IgG immunomics vector 4-fold, although the increase in information content may be less easy to predict because the levels of the antibody sub-classes against a given antigen may be highly correlated (not least because their binding is occurring in competition).

Another approach would be to introduce library elements which bear no structural relationship to the oligopeptides, for example by adding oligosaccharide sub-libraries. It is known that low affinity natural antibodies against oligosaccharide antigens are abundant, temporally stable and vary between individuals because of the large body of work on antibodies against blood group antigens (which are simple carbohydrate structures). Adding sub-libraries of oligosaccharide antigens may thus increase the information content of the immunomic profile with a minimal increase in library complexity. Other chemical antigens could also be included (such as lipids, aromatics and so forth) but the prevalence of natural antibodies to these antigens is less well understood at present.

A suitable change in library design might be to add library elements which provide more resolution in those areas of the broad profile which are known to be of greatest interest (for example, in the example given below, in the first 8 sub-libraries with the hydrophilic amino termini).

Changing the pools of amino acids used during library construction might yield further information from the resulting profile: for example by switching 5 of the amino acids from the “I”-group to the “B”-group and then synthesising a further 256 sub-libraries which are, in some sense, “orthogonal” in composition to the original library might add information content to the immunomic profile at an acceptable increase in library complexity, but any such gains will have to be demonstrated empirically.

H. Diagnostic Methods

An immunomic profile of an individual may also have a diagnostic use. An immunomic profile, for example a profile derived using the DMI techniques described herein can be used to obtain a high density descriptive vector for different individuals which can be used to diagnose the presence of a disease. Most medical conditions or diseases will lead to a change in the immunomic profile of an individual due to responses of the immune system to the particular condition. Some aspects of an immunomic profile may correlate with a particular disease or condition and may, for example be indicative of the cause of the disease or condition or of its effects. Analysis of the immunomic profile of an individual may therefore be used in the diagnosis of a disease in the individual, or to predict a future disease or the susceptibility of the individual to a particular disease. The immunomic profile may also be used to assess the severity or likely severity of the disease in that individual. The methods described herein may also be used to monitor the disease in an individual known to be suffering therefrom. For example, the progression or regression of a disease may be monitored, or the effects of a treatment for the disease may be monitored.

Such a diagnosis may be achieved by deriving standard profiles for individuals whose disease status is known. Pattern recognition techniques may then be used to identify any signatures within the immunomic profiles which are uniquely and reproducibly associated with the presence of the disease or condition. This information can then be used to make predictions about the disease status of other test individuals whose disease status is not yet known.

The presence of, or a susceptibility to, a disease may thus be determined by a method comprising the steps of detecting a plurality of immunoglobulins in a test sample obtained from an individual and then comparing the immunoglobulins detected in the sample, i.e. the immunomic profile of the individual, with known patterns of immunoglobulins or known patterns in the immunomic profile that are associated with the presence or absence of the disease. By making such a comparison, it can be determined whether the individual has, or is likely to develop, the disease in question.

The individual may be any human or animal in which it is desired to form a diagnosis. The detecting step and the production of an immunomic profile for the individual may be carried out by any suitable method, for example using the DMI methods described herein. The comparing step may be carried out by any suitable method. In some cases it may be possible to achieve this manually by inspection of the immunomic profiles. Alternatively, any pattern recognition method may be used, for example those described herein. Suitable pattern recognitions methods may include Principal Component Analysis, Partial Least Squares Discriminant Analysis, genetic computing, a support vector machine, linear discriminant analysis, variable selection algorithms and wavelet decomposition.

Any disease or condition where a correlation is found between disease state and immunomic profile may be diagnosed in this way. Suitable diseases may be those where the immune system plays a key role, or where a variety of factors may contribute to the condition.

Suitable diseases for diagnosis in this way may include, for example, infectious diseases such as those caused by bacteria, fungi, parasites, viruses or prions, parasitic diseases such as those caused by protozoa or worms, inflammatory diseases, autoimmune diseases, genetic diseases, toxic diseases such as those caused by exposure to environmental toxins, conditions caused by injury, malformation, or disuse of parts of the body, nutritional diseases or disorders, neurological disorders, cancer, allergy and heart disease. Particular diseases where the methods described herein may be useful for diagnosis include coronary heart disease, cancers such as luncg cancer and bowel cancer, osteoarthritis, osteoporosis, Alzheimer's disease, Parkinson's disease, Huntingdon's disease, multiple sclerosis, rheumatoid arthritis, systemic lupus erythematosus and endometriosis.

The methods of the invention may be of particular use in the diagnosis of diseases or conditions which it is otherwise difficult to diagnose accurately without use of an invasive procedure.

The diagnostic methods of the invention can be carried out on a test sample which has been obtained from the patient. Any test sample that comprises immunoglobins may be used in such a method. For example, the test sample may be blood, serum, plasma, tissue sample or cerebrospinal fluid.

Kits are also envisaged for use in the methods described herein, for example for use in obtaining an immunomic profile for an individual or for use in a diagnostic method. A suitable kit will comprise components that would be used in such a method. For example a kit may comprise a plurality of antigens or mixtures of antigens wherein each antigen or antigen mixture comprises a tag, together with one or more labelled antibodies capable of specifically binding to immunoglobulins. Any antigens, mixture of antigens or library of antigens as described herein may be used in such a kit. Similarly, any labelled antibodies described herein may be used. A preferred kit may comprise a library of peptides which has been produced as described herein using the amino acid grouping shown below in Table 5, wherein each mixture of peptides within the library is tagged with aluminium barcodes. A preferred kit may also comprise a labelled antibody capable of specifically detecting IgG.

### EXAMPLES

### Example 1

### A Proteomic Analysis of Human Serum Using a Small Antibody Library Aluminium Bar-Code Tags and a Fluorescein Labelled Reference Sample

In the first step, an antibody library suitable for use in DMI was generated. For this pilot demonstration of the invention, the library was constructed by obtaining quantities of purified antibodies against human serum components from a range of manufacturers. Each of the antigens to be studied was included in the library just once, and as a result the library had the ideal characteristic for DMI libraries of very low redundancy.

For this experiment, thirty eight different antibodies were selected. Thirty-four were against distinct serum components (see Table 1). The remaining 4 were control antibodies of the same species as the 34 antibodies, but with epitopes selected to be absent from the reference sample. The 34 serum components to be detected in this experiment ranged in abundance from albumin (˜30 mg/ml) to IL-1b (100 pg/ml). However, for three of the antibodies against the least abundant components (anti-Htp24gag, anti-soluble selectin and anti-IL1b) no signal was detected in the reference sample and consequently no data was obtained from these tags. The least abundant protein to be robustly detected in our experiment was TGF-beta (˜30 ng/ml), representing a working dynamic range for DMI of approximately 1 million fold. Since each antibody was purchased separately, they were available in 38 separate containers, allowing them to be dispensed at an antibody concentration of 1 mg/ml in phosphate-buffered saline into wells of a microtitre plate.

TABLE 1

| Tag | Antigen | Antibody | Species | CVar |
|---|---|---|---|---|
| 1 | α2-macroglobulin | Biogenesis 5850-0004 | Sheep IgG | 3.8 |
| 2 | α1-antitrypsin | Calbiochem 178260 | Mouse IgG2a | 2.1 |
| 3 | ApoAI | Calbiochem 178422 | Rabbit IgG | 7.2 |
| 4 | ApoB | Calbiochem 178426 | Rabbit IgG | 11.4 |
| 5 | ApoE | Biogenesis 0650-2054 | Mouse IgG1 | 6.8 |
| 6 | β2-microglobulin | Sigma M7398 | Mouse IgG1 | 2.3 |
| 7 | CICP | Quidel 1M0622 | Rabbit IgG | 2.2 |
| 8 | Fibrinogen | Biogenesis 4440-8004 | Sheep IgG | 3.0 |
| 9 | HIV1p24gag | ARP ARP313 | Mouse IgG | — |
| 10 | ICAM-1 | Serotec MCA532 | Mouse IgG1 | 17.6 |
| 11 | Ig Kappa LC | Bionostics M03010 | Mouse IgG1 | 2.6 |
| 12 | IgA | Bionostics M26012 | Mouse IgG1 | 2.4 |
| 13 | IgD | Bionostics M01014 | Mouse IgG1 | 2.9 |
| 14 | IgE | Bionostics M38041 | Mouse IgG1 | 8.1 |
| 15 | IGF-1 | Serotec MCA520 | Mouse IgG1 | 2.3 |
| 16 | IL1β | R&D Systems | Mouse IgG1 | — |
| 17 | Lp(a) | Immunoscientific | Sheep IgG | 4.5 |
| 18 | MMP9 | Chemicon AB805 | Rabbit IgG | 3.5 |
| 19 | Myeloperoxidase | NeoRX NR-ML-5 | Mouse IgG | 2.6 |
| 20 | Osteopontin | Hoyer 1826-1283 | Rabbit IgG | 3.3 |
| 21 | PAI-1 (free) | Progen TC21173 | Mouse IgG1 | 6.9 |
| 22 | PAI-1 (complex) | Mol Innovations | Mouse IgG1 | 2.5 |
| | | MA14D5 | | |
| 23 | PAI-2 | American Diagnostic | Mouse IgG2a | 2.7 |
| | | #3750 | | |
| 24 | PDGFAA/AB | UBI #06-130 | Rabbit IgG | 4.6 |
| 25 | Selectin E/P | R&D Systems BBA1 | Mouse IgG1 | — |
| 26 | Serum Albumin | Calbiochem 126582 | Rabbit IgG | 3.8 |
| 27 | SHBG | Biogenesis 8280-0108 | Mouse IgG1 | 2.6 |
| 28 | TGF-β1 | R&D Systems BDA19 | Chicken IgG | 5.0 |
| 29 | TGF-LTBP | R&D Systems Mab39 | Mouse IgG | 4.7 |
| 30 | Thrombospondin | Biogenesis 8835-0004 | Mouse IgG1 | 2.3 |
| 31 | TIMP-2 | Biogenesis 9013-2609 | Sheep IgG | 3.3 |
| 32 | TPA | American Diagnostic | Goat IgG | 2.4 |
| | | #387 | | |
| 33 | UPA | Accurate YMPS75 | Goat IgG | 2.9 |
| 34 | VWF | Dako A082 | Rabbit IgG | 4.6 |
| 35 | Collagen-II | NIHDHSB CII-C1 | Mouse IgG | — |
| 36 | NR58-3.14.3 | Affiniti ARP063/AF | Rabbit IgG | — |
| 37 | Salicylate | Cortex CR1041SP | Sheep IgG | — |
| 38 | PPAR-alpha | Santa Cruz sc1985 | Goat IgG | — |

Table 1:
The antibodies that were selected to generate the small manual DMI library are shown above.
‘Tag’ numbers represent the position of the library component in the output vector (and is not the code of the tag, which is more complex).
‘Antigen’ represents the known serum component that the antibody binds to.
‘Antibody’ represents the source of the particular antibody used.
‘Species’ is the species of the immunoglobulin fraction used.
‘Cvar’ is the coefficient of variation for reading multiple tags of the same code in the same experiment. The Cvar is not given for HIVp24gag, ICAM-1 or SelectinE/P because these antigens were below the detection limit of the assay in our reference sample.

This small antibody library was then tagged using aluminium barcode tags. The tags were activated to promote non-covalent protein binding, then mixed with the antibodies: a different bar code was mixed with each component of the antibody library. The tags and antibodies were sealed and incubated overnight to allow the bar code tags to become fully coated in antibody molecules. All the tagged antibodies are then pooled into a single tube, and wash them by gentle ultrafiltration with an excess of phosphate-buffered saline, and resuspended at a known tag concentration (e.g. 1 million individual tags per ml).

In the second step, the labelled reference sample was prepared. Approximately 2 ml of pooled serum from 15 healthy volunteers was extensively dialysed against 100 mM sodium carbonate buffer pH9 (to remove free amino acids that would prevent the reaction between proteins and the fluorescein isothiocyanate (FITC), as well as to adjust the pH to the optimum for FITC labelling). FITC dissolved in DMSO was then added to the dialysed serum at approximately a molar ratio of approximately 10:1 (serum contains 70 mg/ml protein of average molecular mass 50,000 Da, which is equivalent to a concentration of −1.4 mM; therefore FITC is added to a final concentration of 15 mM. To 2 ml of serum, we added 200 μl of 150 mM stock FITC in DMSO).

The labelling reaction was left to run overnight at 4° C. with constant mixing. The reaction was then terminated by addition of 1/10^{th} volume (220 μl) of 1M glycine pH 7, The excess glycine rapidly reacts with any free FITC remaining and hence terminates the reaction The resulting protein mixture is then separated from the unreacted fluorescein:glycine conjugate by column chromatography. A sephadex G25 column (10 ml bed volume) was equilibrated in phosphate-buffered saline, then loaded with the labelled serum sample. The protein component rapidly passes through the column and is collected and retained, while the low molecular weight salts (including the fluorescein) pass much more slowly through the column and are discarded. The separation can be monitored by flowing the column eluate through a dual-wavelength spectrophotometric detector set at 280 nm (to observe protein) and 490 nm (to observe fluorescein). The trace obtained is shown in FIG. 2.

The labelled protein eluate from the column was then concentrated using a centrifugal ultraconcentrator (Millipore) with a nominal 3 kDa cut-off filter membrane until it was reduced in volume to approximately 1 ml—half the original volume of pooled serum. The total protein concentration of this sample was then tested using a Coomassie Plus protein assay (Pierce) with serum albumin as the standard. In our experiment, the protein concentration was 121 mg/l representing a recovery of 86% during the labelling and chromatography steps. An appropriate volume of phosphate-buffered saline was then added to return the total protein concentration of the labelled reference sample to that of the original pooled serum. In our experiment, 730 μl of buffer was added to return the total protein concentration to 70 mg/ml. This procedure prepared 1.73 ml of labelled reference sample, sufficient for approximately 100 separate assays. The same procedure, however, can be used to prepare much larger batches of reference sample.

In the third step, we performed the actual DMI procedure. In a V-bottom microtitre plate, 2011 aliquots of the labelled reference sample were dispensed. Next, 20 μl of each test was sample was added to each well—the test samples were undiluted human serum samples, including the 15 samples that had been pooled to create the reference sample pool. The plate was sealed and mixed. Next 10 μl of the tagged DMI antibody library (containing about 10,000 individual tags—we aim to add between 10 and 200 times as many individual tags are there are discrete components to the library to increase the likelihood that at least one of every tag is included in the mixture) was dispensed into each well. The plate was again sealed, mixed and then incubated at room temperature for 15 minutes with constant agitation. At the end of the experiment, 150 μl of phosphate buffered saline was added to terminate the reaction by dilution.

In the final step, each reaction in turn was passed through a flow cytometer. For large scale DMI experiments, this can be performed using a robotic autosampler, but for this smaller scale pilot experiment, each reaction in turn was transferred to a FACS tube (Becton-Dickinson) and manually sampled. For each tube 5,000 events were captured (representing 5,000 distinct individual tags). As each tag passed through the laser beam, the time profile of the forward-scatter pulse was decoded to give the binary representation of the tag code. Simultaneously, the FL1 pulse height read at 90° to the incident beam, was taken to represent the amount of labelled protein bound to the tagged antibody. Each pair of numbers (tag code, bound label) were recorded for all 5,000 events. Thereafter, the events were grouped by tag code, and the average bound label for each group of identical codes was calculated. The output from this experiment was a vector with 38 values in tag code order for each of the samples analysed. The results are shown in Table 2 and FIG. 3. These profiles represent a proteomic profile for each of the individuals tested, and can be used for various investigation or analytical purposes.

In this example, we noted that several of the individuals had elevated levels of the proteins bound to tags 8 and 21 (this is represented by the lower values in Table 2, since high levels of a protein in the test sample reduces the amount of labelled protein from the reference sample which binds to the tagged antibody). These tags had antibodies to fibrinogen and PAI-1 respectively. Since these proteins are both known to be positive acute phase reactants (that is, there levels are known to be elevated during infections), we conclude that these individuals are likely to have been suffering from a minor infection, such as the common cold, at the time the blood sample was drawn.

We have performed a fall analysis of the sources of variation in the data vector obtained (Tables 1 & 2). Firstly, we have assessed the analytical reproducibility of the method (Cvar(anal)) calculated from the range of fluorescence readings from different tags with the same code in the same experiment. The analytical reproducibility is excellent (below 5% for most tags, superior to individual immunoassays). Furthermore, the Cvar(anal) is unaffected by the abundance of antigen, being similar for albumin and fibrinogen to TGF-beta and PAI-1.

Furthermore, five of the samples tested were replicate aliquots from the same bleed (P1 to P5, shaded in Table 2). This allows the repeated measures reproducibility (Cvar(rm)) to be assessed. The Cvar(rm) is reported with the analytical variation (Cvar(anal)) subtracted. The median Cvar(rm) for all 31 antibodies for which a signal was detected in the reference sample was 2.7% (range 2.1% to 17.6%) which is slightly inferior to the most robust analytical methods such as NMR for metabonomics (1-2%), but considerably better than any existing proteomic methods, including 2D gel electrophoresis or protein chip microarrays (10-20%).

TABLE 2

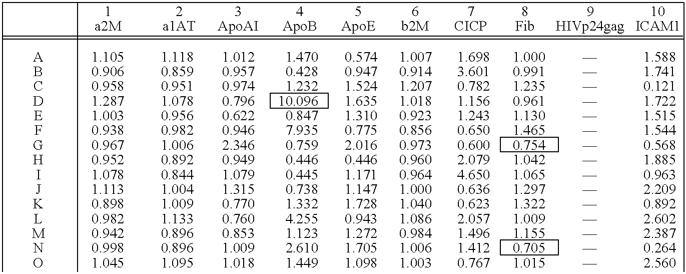

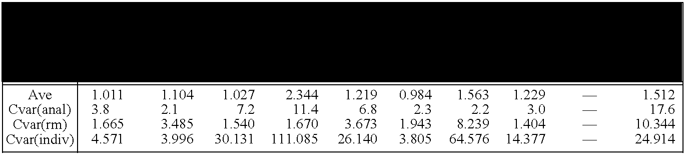

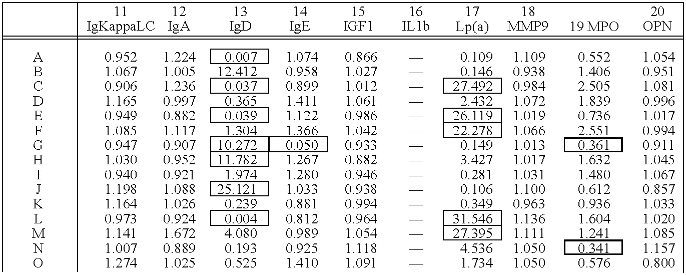

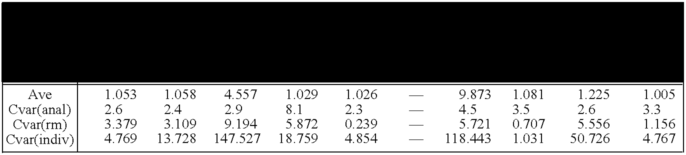

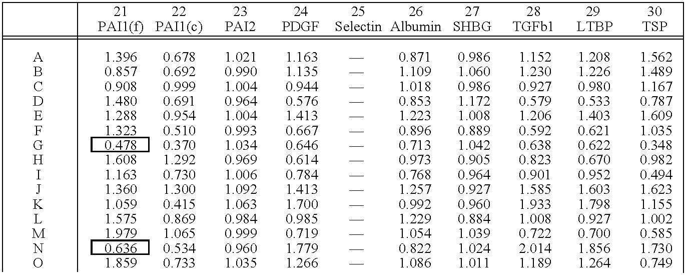

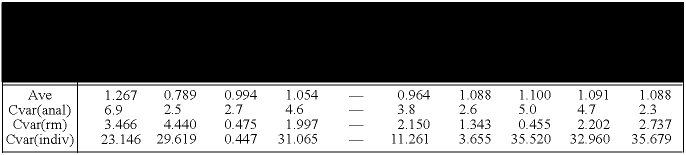

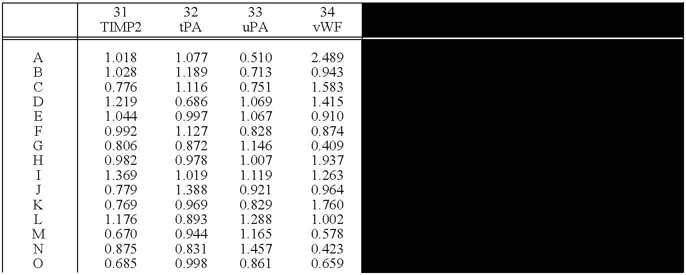

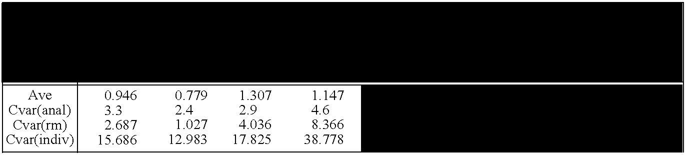

### Example 2

### Generation of a Large Scale DMI Antibody Library from an Unselected Phage Display Library with Very High Coverage

In example 1, we used a manually constructed small DMI antibody library to illustrate the principle of the approach. However, as with any megaplex technology capable of managing thousands of analytes in parallel, the power of the approach increases with the size of the library. It is not feasible to construct libraries larger than 100 or so components by the manual method, so an alternative is required for large libraries. Furthermore, a manually constructed library will only represent “known” antigens (that is, ones already known or suspected to be present in the test samples). In contrast, a library generated by sub-selection from a phage-display library will be both much larger and likely to contain antibodies to components of the test sample that have never previously been identified.

The prerequisite for successful generation of a large DMI library is a master phage display library with very broad coverage. The higher the number of independent clones composing the master library, the better the resulting DMI library that can be sub-selected from it. The master library can be constructed by any of the methods well known in the art, and examples include the CAT library that contains approximately 10¹³ independent clones, representing at least 10 times the immune diversity of a human subject.

To prepare the large DMI library, an unlabelled aliquot of the reference sample (in our case, the pooled serum from 15 healthy individuals) was coated onto tissue culture plastic (high protein binding plastic) at low protein density (approximately 10 μg protein per cm²) to ensure that all, or almost all of the proteins present in the reference sample were bound. A total surface area of about 1,000 cm² was prepared in this way (with 10 mg total protein). The master phage library was then expanded and passed over the plate surface at room temperature for 30 minutes. Unbound phage were washed away thorough with phosphate buffered saline containing 0.1% Tween 20.

The positively selected phage were then released, and the population again expanded. In the second step, the reference sample protein was coated onto tissue culture plastic at very high protein density (10 mg of protein per cm²). With the number of protein binding sites on the plastic severely limiting, many of the rarer proteins will not be represented at all on the plate, while the abundant proteins will be highly represented. The selected phage were then exposed to this surface for 30 minutes at room temperature, and this time the unbound phage were retained and the bound phage were discarded.

This process was repeated a number of times, expanding the phage population, then applying positive selection, expanding the population and performing negative selection and so forth. As the process continued, the redundancy of the library falls, and the bias towards abundant antigens in the reference sample also falls. The bias was monitored as the selection process was iterated: four purified antigens (two abundant (fibrinogen and albumin) and two rare (TGF-beta and PAI-1)) were coated onto ELISA plate wells in 100 mM sodium carbonate pH9 at 4° C. overnight, then washed and blocked using 5% sucrose/5% Tween in phosphate buffered saline. After washing the wells again (in phosphate buffered saline+0.1% Tween) a serial dilution of the selected library was applied to each antigen. This was allowed to bind for 30 minutes at room temperature, then the wells were washed, and the bound phage detected with an anti-phage coat protein antibody labelled with horseradish peroxidase. After further washes, the amount of bound enzyme was quantitated using the substrate K-BLUE. The dilution of the library that yielded half maximal signal on each antigen was then determined (with undiluted library assigned the arbitrary concentration of 1 unit). The bias of the library was calculated as the mean for the two abundant antigens divided by the mean for the two rare antigens. The bias of the subselected DMI library as we performed four iterations of positive and negative selection are shown in FIG. 4.

This example demonstrates that it is possible to generate a large DMI library with low redundancy and low bias which could be limiting dilution cloned in microtitre plates to generate a tagged library similar to the one used in example 1 but with 10,000 to 100,000 individual components.

### Example 3

### Immunomics Using a Small-Scale Carbohydrate Antigen Library

As the first step, an antigen library must be assembled. For this pilot-scale experiment, the library was manually constructed by dispensing individually synthesised and purified carbohydrate antigens into wells of a 96 well plate. Twenty four different oligosaccharide sequences were commercially available (Glycorex) coupled to serum albumin (Table 3). Serum albumins (bovine or human origin) without any carbohydrate attached were used as control library components dispensed into 2 further wells. In each well, approximately 100 μg of protein/oligosaccharide conjugate was dispensed.

TABLE 3

| Tag | Antigen | Conjugate | Carrier | CVar |
|---|---|---|---|---|
| 1 | Glcβ-O-spacer | B-1001 | BSA | 2.1 |
| 2 | Galβ-O-spacer | B-1002 | BSA | 2.3 |
| 3 | Manα-O-spacer | B-1003 | BSA | 1.9 (M) |
| 4 | Galβ1-4Glcβ-O-spacer | B-1004 | BSA | 4.8 |
| 5 | Galβ1-4GlcNAcβ-O-spacer | B-1005 | BSA | 3.0 |
| 6 | Glcα1-6Glcα1-4Glcβ1-4Glcβ-O- | B-1007 | BSA | — |
| | spacer | | | |
| 7 | Galα1-4Galβ1-4G1cβ-O-spacer | B-1017 | BSA | 2.2 |
| 8 | Galα1-4Galβ1-4GlcNAcβ-O- | B-1010 | BSA | 2.6 |
| | spacer | | | |
| 9 | Galα1-4Galβ-O-spacer | B-1011 | BSA | 2.1 |
| 10 | Galβ1-3GlcNAcβ-O-spacer | B-1012 | BSA | 2.4 |
| 11 | Di-Manα1-6(α1-3)Manα-O-spacer | B-1014 | BSA | — |
| 12 | GalNAcβ1-3Galα-O-spacer | B-1015 | BSA | 2.7 |
| 13 | GalNAcβ1-4Galβ-O-spacer | B-1016 | BSA | 2.2 |
| 14 | GalNAcβ-O-spacer | B-1018 | BSA | 2.1 |
| 15 | GalNAcα1-3(Fucα1-2)Galβ-O- | B-1019 | BSA | 6.1 |
| | spacer | | | |
| 16 | Galα1-3(Fucα1-2)Galβ-O-spacer | B-1020 | BSA | 4.4 |
| 17 | Galα1-3Gal-O-spacer | B-1008 | BSA | 2.4 |
| 18 | Galα1-3Galβ1-4GlcNAcβ-O- | B-1009 | BSA | 2.5 |
| | spacer | | | |
| 19 | Galα-O-spacer | H-1021 | HSA | 3.3 |
| 20 | Galα1-2Gal-O-spacer | H-1022 | HSA | 3.2 |
| 21 | Galα1-3Galβ1-4GlcNAcβ1- | H-1025 | HSA | 2.3 |
| | 3Galβ1-4Glc-O-spacer | | | |
| 22 | Galα1-4Gal-O-spacer | H-1026 | HSA | 2.8 |
| 23 | Galα1-3GalNAcα-O-spacer | H-1030 | HSA | 3.7 |
| 24 | Galβ1-3GalNAcα-O-spacer | H-1031 | HSA | 3.2 |
| 25 | None | Glycorex | BSA | 6.9 (M) |
| 26 | None | Glycorex | HSA | — |
| Table 3: | | | | |
| The glycoconjugate antigens that were selected to generate the small manual DMI library for immunomics are shown above. | | | | |
| ‘Tag’ numbers represent the position of the library component in the output vector (and is not the code of the tag, which is more complex). | | | | |
| ‘Antigen’ represents the carbohydrate sequence in the conjugate. | | | | |
| ‘Conjugate’ represents the source of the particular conjugate used - all the catalog codes refer to the Glycorex catalog. | | | | |
| ‘Carrier’ indicates the carrier protein to which the carbohydrate antigens are conjugated, where BSA represents bovine serum albumin and HSA represents human serum albumin. Unconjugated aliquots of the same batch of these proteins were used as controls on tags 25 and 26. | | | | |
| ‘Cvar’is the coefficient of variation for reading multiple tags of the same code in the same experiment. The Cvar is the mean of the Cvar for the pan-IgG (FITC) vector and the IgM (rPE) vector, except where stated when too little IgG bound to the antigen to be quantified. A dash indicates that neither Ig class bound to the antigen to any significant degree. Note that the Cvar reported is the mean from 15 different individuals, to reflect | | | | |
| # the varying signal bound to each tag which results in a varying analytical CVar from individual to individual (in contrast to Table 1, where the analytical Cvar depends on the average signal from all of the individuals, represented by the reference sample). | | | | |

The antigen library was then tagged, using aluminium bar code tags, exactly as described in example 1 for an antibody library. Since the oligosaccharide antigens were carried on protein scaffolds, the same chemistry that is used to bind antibody protein to the aluminium, also achieves attachment of the oligosaccharide/protein conjugates. A different pool of aluminium bar coded tags was dispensed into each well (about 10⁴ individual tags in each pool). At the end of the tagging reaction, the tags were harvested and washed in phosphate-buffered saline by gentle ultrafiltration, and resuspended in 100 μl per well of phosphate-buffered saline. All the wells were then combined to yield approximately 2 ml of library containing a total of 2×10⁵ individual tags at 100,000 tags per ml.

In the second step, serum samples from 15 healthy volunteers were dispensed at 20 μl per sample directly into V-bottom microtitre plate wells. 20 μl of the library was then added (approximately 2,000 individual tags, representing a 100-fold excess over the number of individual components of the library). Non-ionic detergent (Tween 20 at 0.1% vol/vol final concentration) was also added to the reaction mixture to improve the specificity of antibody binding, and lower the background. The plate was then sealed and the reaction mixed thoroughly, and incubated at room temperature with continual agitation for 15 minutes.

At the end of the incubation, the tags were harvested and washed by gentle ultrafiltration over a vacuum manifold, and phosphate-buffered saline containing 0.1% Tween 20 was used throughout as the wash solution. The beads were then resuspended in 50 μl of phosphate-buffered saline with 0.1% Tween 20 and each of the WHO standard mouse monoclonal anti-human Ig class specific antibodies labelled with a different fluorochrome. For this experiment, we used the anti-pan IgG antibody labelled with FITC and the anti-IgM antibody labelled with TRITC. Each of the detection antibodies was present at 5 μg/ml final concentration. The plate was then sealed and mixed, before being incubated at room temperature with continual agitation for 15 minutes.

As the third step, for detection of the antibodies a fluorescence microscope was used. The reaction from each well in turn was dispensed onto a standard glass microscope slide in a well about 1 cm in diameter inscribed using a PAP pen. A coverslip was placed over the slide and sealed to prevent evaporation using clear nail varnish. The slide was then placed under a fluorescence microscope, and the bar coded tags located, one at a time, under direct illumination. As each tag was located, its binary code was read and logged. The amount of fluorescence in the fluorescein channel and rhodamine channel were then determined using an automated filterwheel changer. The two separate fluorescence readings were then recorded together with the bar code for each tag. Where more than one tag was located in each reaction with the same binary code, the fluorescence readings from the two (or more) identical tags were averaged prior to reporting the immunomic profile vector. Approximately 500 individual tags were read for each reaction. Using a manual microscope system, this take approximately one hour per sample analysed. However, automated systems do exist for reading the fluorescence bound to each bar coded tag under a microscope. Alternatively, the tags could be read using an appropriate flow cytometer (see example 1).

TABLE 4

| | | | | | | | | | 5 | | 6 | | 7 | |
|---|---|---|---|---|---|---|---|---|---|---|---|---|---|---|
| | 1 | | 2 | | 3 | | 4 | | Nac-lacA | | GlyStor | | Pk | |
| A | 3 | 141 | 2 | 0 | 0 | 0 | 0 | 10 | 35 | 23 | 0 | 0 | 140 | 103 |
| B | 21 | 116 | 1 | 1 | 0 | 13 | 1 | 6 | 24 | 57 | 0 | 0 | 2 | 7 |
| C | 14 | 30 | 6 | 39 | 0 | 0 | 4 | 13 | 40 | 108 | 0 | 0 | 107 | 410 |
| D | 13 | 45 | 2 | 42 | 0 | 0 | 0 | 2 | 36 | 7 | 0 | 0 | 119 | 125 |
| E | 11 | 20 | 6 | 33 | 0 | 0 | 3 | 7 | 48 | 43 | 0 | 0 | 0 | 68 |
| F | 1 | 113 | 3 | 14 | 0 | 3 | 282 | 44 | 35 | 151 | 0 | 0 | 1 | 31 |
| G | 22 | 52 | 4 | 552 | 1 | 2 | 25 | 15 | 53 | 52 | 33 | 244 | 75 | 134 |
| H | 7 | 30 | 8 | 2 | 0 | 0 | 4 | 15 | 55 | 70 | 0 | 0 | 142 | 99 |
| I | 23 | 43 | 3 | 1 | 0 | 1 | 0 | 10 | 73 | 189 | 0 | 0 | 53 | 86 |
| J | 2 | 94 | 2 | 10 | 3 | 1 | 1 | 27 | 35 | 68 | 0 | 0 | 238 | 113 |
| K | 21 | 32 | 1 | 11 | 0 | 0 | 96 | 27 | 101 | 200 | 0 | 1 | 62 | 321 |
| L | 5 | 48 | 2 | 15 | 0 | 0 | 94 | 54 | 20 | 84 | 0 | 0 | 201 | 231 |
| M | 5 | 39 | 2 | 12 | 0 | 0 | 0 | 11 | 97 | 43 | 0 | 0 | 137 | 371 |
| N | 11 | 34 | 4 | 6 | 0 | 0 | 68 | 43 | 28 | 42 | 0 | 0 | 142 | 122 |
| O | 6 | 37 | 4 | 6 | 0 | 0 | 1 | 31 | 28 | 46 | 0 | 0 | 221 | 960 |
| P1 | 3 | 33 | 6 | 13 | 0 | 1 | 4 | 2 | 37 | 68 | 0 | 0 | 53 | 2 |
| P2 | 3 | 42 | 5 | 15 | 0 | 0 | 3 | 1 | 42 | 60 | 0 | 0 | 68 | 2 |
| P3 | 4 | 47 | 5 | 19 | 0 | 2 | 3 | 2 | 44 | 68 | 0 | 0 | 17 | 2 |
| P4 | 3 | 37 | 5 | 15 | 0 | 1 | 3 | 2 | 42 | 61 | 0 | 1 | 69 | 2 |
| P5 | 4 | 39 | 5 | 16 | 0 | 1 | 4 | 2 | 38 | 67 | 0 | 0 | 60 | 2 |
| Median | 11 | 43 | 3 | 11 | 0 | 0 | 3 | 15 | 36 | 57 | 0 | 0 | 119 | 122 |
| Cvar(anal) | 2.2 | 2.1 | 2.1 | 2.5 | — | 11.9 | 5.5 | 4.1 | 3.3 | 2.7 | — | — | 2.2 | 2.2 |
| Cvar(rm) | 13.9 | 11.2 | 6.5 | 11.5 | — | 49.5 | 10.6 | 9.0 | 4.0 | 3.4 | — | — | 37.8 | 8.2 |
| Cvar(indiv) | 54 | 52 | 52 | 267 | — | 185 | 180 | 63 | 46 | 68 | — | — | 30 | 103 |
| | 8 | | 9 | | | | | | | | | | | |
| | P1 | | EColiR | | 10 | | 11 | | 12 | | 13 | | 14 | |
| A | 29 | 32 | 87 | 454 | 3 | 4 | 0 | 0 | 6 | 8 | 5 | 9 | 1 | 4 |
| B | 136 | 242 | 6 | 59 | 3 | 8 | 0 | 1 | 5 | 10 | 2 | 19 | 13 | 4 |
| C | 62 | 87 | 41 | 0 | 1 | 6 | 0 | 3 | 8 | 153 | 6 | 5 | 21 | 32 |
| D | 94 | 109 | 15 | 5 | 6 | 3 | 0 | 0 | 5 | 33 | 7 | 9 | 1 | 2 |
| E | 211 | 581 | 5 | 15 | 2 | 20 | 0 | 0 | 4 | 6 | 4 | 22 | 2 | 2 |
| F | 176 | 146 | 46 | 5 | 1 | 2 | 0 | 0 | 6 | 9 | 3 | 14 | 0 | 3 |
| G | 74 | 102 | 2 | 3 | 7 | 3 | 0 | 0 | 4 | 29 | 5 | 17 | 1 | 4 |
| H | 33 | 78 | 65 | 41 | 2 | 4 | 0 | 0 | 4 | 23 | 4 | 7 | 3 | 2 |
| I | 71 | 32 | 7 | 363 | 4 | 6 | 0 | 0 | 4 | 16 | 5 | 8 | 15 | 20 |
| J | 41 | 293 | 45 | 361 | 2 | 3 | 0 | 0 | 6 | 12 | 5 | 13 | 3 | 4 |
| K | 27 | 32 | 4 | 4 | 8 | 36 | 0 | 0 | 14 | 12 | 4 | 13 | 1 | 2 |
| L | 63 | 93 | 13 | 150 | 1 | 6 | 0 | 0 | 8 | 9 | 4 | 8 | 1 | 6 |
| M | 91 | 57 | 96 | 18 | 11 | 7 | 0 | 0 | 10 | 13 | 2 | 9 | 3 | 10 |
| N | 60 | 178 | 12 | 1 | 9 | 4 | 0 | 0 | 4 | 51 | 3 | 5 | 4 | 20 |
| O | 100 | 68 | 0 | 1 | 1 | 21 | 0 | 0 | 2 | 9 | 0 | 2 | 3 | 1 |
| P1 | 103 | 143 | 56 | 21 | 3 | 6 | 0 | 0 | 4 | 38 | 1 | 10 | 4 | 13 |
| P2 | 97 | 157 | 52 | 16 | 3 | 5 | 0 | 0 | 3 | 32 | 2 | 10 | 3 | 17 |
| P3 | 104 | 155 | 48 | 18 | 1 | 5 | 0 | 0 | 4 | 40 | 1 | 12 | 5 | 14 |
| P4 | 109 | 155 | 47 | 21 | 3 | 7 | 0 | 0 | 4 | 31 | 1 | 13 | 5 | 11 |
| P5 | 102 | 160 | 46 | 18 | 2 | 3 | 0 | 0 | 3 | 33 | 1 | 11 | 3 | 16 |
| Median | 71 | 93 | 13 | 15 | 3 | 6 | 0 | 0 | 5 | 12 | 4 | 9 | 3 | 4 |
| Cvar(anal) | 2.2 | 3.0 | 2.0 | 2.3 | 2.7 | 2.1 | — | — | 3.0 | 2.5 | 2.1 | 2.2 | 2.1 | 2.1 |
| Cvar(rm) | 2.0 | 1.2 | 6.3 | 9.2 | 34.6 | 26.4 | — | — | 12.2 | 8.9 | 35.2 | 9.9 | 22.9 | 14.7 |
| Cvar(indiv) | 59 | 97 | 100 | 149 | 44 | 78 | — | — | 35 | 130 | 7 | 40 | 106 | 101 |
| | 15 | | 16 | | 17 | | 18 | | | | | | 21 | |
| | A | | B | | Di-aGal | | Tri-aGAl | | 19 | | 20 | | Pentagal | |
| A | 252 | 557 | 293 | 296 | 81 | 133 | 108 | 92 | 3 | 26 | 6 | 59 | 77 | 68 |
| B | 198 | 1098 | 461 | 607 | 119 | 62 | 830 | 456 | 4 | 10 | 14 | 21 | 465 | 696 |
| C | 1 | 127 | 569 | 113 | 67 | 31 | 46 | 30 | 1 | 22 | 84 | 32 | 43 | 881 |
| D | 438 | 231 | 213 | 458 | 33 | 29 | 138 | 44 | 2 | 37 | 25 | 13 | 18 | 324 |
| E | 0 | 15 | 147 | 1436 | 47 | 39 | 1160 | 124 | 4 | 467 | 146 | 148 | 436 | 245 |
| F | 0 | 38 | 336 | 209 | 82 | 108 | 32 | 161 | 5 | 34 | 5 | 54 | 58 | 89 |
| G | 69 | 1664 | 0 | 3 | 16 | 19 | 40 | 67 | 6 | 40 | 26 | 12 | 34 | 58 |
| H | 7 | 11 | 289 | 469 | 46 | 72 | 242 | 287 | 2 | 20 | 3 | 34 | 82 | 39 |
| I | 552 | 208 | 119 | 991 | 13 | 84 | 161 | 132 | 5 | 11 | 27 | 99 | 65 | 218 |
| J | 1 | 4 | 460 | 526 | 35 | 127 | 149 | 536 | 4 | 30 | 3 | 12 | 12 | 94 |
| K | 0 | 46 | 238 | 672 | 12 | 27 | 67 | 87 | 6 | 16 | 30 | 38 | 29 | 475 |
| L | 297 | 794 | 301 | 219 | 104 | 75 | 553 | 148 | 5 | 44 | 2 | 102 | 25 | 264 |
| M | 0 | 43 | 262 | 816 | 10 | 127 | 69 | 1317 | 5 | 27 | 6 | 54 | 24 | 405 |
| N | 0 | 3 | 290 | 655 | 64 | 40 | 81 | 562 | 3 | 12 | 1 | 44 | 45 | 78 |
| O | 360 | 288 | 452 | 200 | 422 | 135 | 409 | 589 | 7 | 17 | 335 | 422 | 5 | 482 |
| P1 | 278 | 462 | 221 | 627 | 64 | 117 | 162 | 442 | 13 | 20 | 5 | 49 | 70 | 268 |
| P2 | 256 | 398 | 292 | 556 | 82 | 109 | 178 | 409 | 11 | 23 | 7 | 42 | 73 | 242 |
| P3 | 292 | 450 | 165 | 691 | 73 | 102 | 155 | 471 | 11 | 27 | 6 | 27 | 66 | 209 |
| P4 | 291 | 426 | 244 | 603 | 79 | 116 | 159 | 477 | 12 | 26 | 6 | 46 | 71 | 253 |
| P5 | 258 | 511 | 268 | 617 | 89 | 92 | 177 | 504 | 10 | 26 | 5 | 48 | 84 | 257 |
| Median | 7 | 127 | 290 | 469 | 47 | 72 | 138 | 148 | 4 | 26 | 14 | 44 | 43 | 245 |
| Cvar(anal) | 5.5 | 6.7 | 4.2 | 4.6 | 2.3 | 2.4 | 2.4 | 2.6 | 3.6 | 3.0 | 3.2 | 3.3 | 2.3 | 2.4 |
| Cvar(rm) | 0.8 | 2.7 | 16.2 | 3.3 | 9.9 | 7.3 | 4.0 | 5.3 | 6.4 | 8.8 | 11.2 | 18.0 | 7.0 | 6.8 |
| Cvar(indiv) | 125 | 134 | 30 | 66 | 120 | 48 | 116 | 103 | 31 | 200 | 172 | 114 | 146 | 77 |

| | | | | |
|---|---|---|---|---|
| | | | 25 | 26 |
| 22 | 23 | 24 | BSA | HSA |

| A | 37 | 311 | 4 | 17 | 19 | 177 | 0 | 0 | 0 | 0 |
|---|---|---|---|---|---|---|---|---|---|---|
| B | 14 | 135 | 9 | 39 | 32 | 31 | 0 | 3 | 0 | 0 |
| C | 13 | 1915 | 51 | 194 | 51 | 31 | 0 | 17 | 0 | 0 |
| D | 4 | 7 | 37 | 50 | 7 | 16 | 0 | 2 | 0 | 0 |
| E | 107 | 608 | 68 | 552 | 92 | 166 | 0 | 1 | 0 | 0 |
| F | 20 | 6 | 13 | 318 | 14 | 20 | 0 | 9 | 0 | 0 |
| G | 74 | 12 | 16 | 47 | 97 | 14 | 0 | 2 | 0 | 0 |
| H | 22 | 15 | 5 | 104 | 39 | 8 | 0 | 3 | 0 | 0 |
| I | 40 | 4 | 147 | 38 | 33 | 144 | 46 | 191 | 0 | 0 |
| J | 34 | 299 | 22 | 113 | 107 | 307 | 0 | 1 | 0 | 0 |
| K | 11 | 10 | 18 | 53 | 39 | 59 | 0 | 0 | 0 | 0 |
| L | 19 | 1 | 12 | 65 | 29 | 39 | 0 | 3 | 0 | 0 |
| M | 4 | 4 | 11 | 76 | 53 | 35 | 0 | 1 | 0 | 0 |
| N | 29 | 2 | 109 | 262 | 126 | 34 | 0 | 1 | 0 | 0 |
| O | 22 | 54 | 154 | 175 | 84 | 126 | 0 | 0 | 0 | 0 |
| P1 | 4 | 14 | 38 | 209 | 26 | 172 | 0 | 3 | 0 | 0 |
| P2 | 3 | 11 | 46 | 248 | 22 | 174 | 0 | 2 | 0 | 0 |
| P3 | 4 | 10 | 52 | 238 | 19 | 188 | 0 | 2 | 0 | 0 |
| P4 | 3 | 13 | 59 | 258 | 25 | 169 | 0 | 3 | 0 | 0 |
| P5 | 4 | 12 | 54 | 250 | 23 | 170 | 0 | 4 | 0 | 0 |
| Median | 22 | 12 | 18 | 76 | 39 | 35 | 0 | 2 | 0 | 0 |
| Cvar(anal) | 2.7 | 2.9 | 2.8 | 4.6 | 3.4 | 3.0 | — | 6.9 | — | — |
| Cvar(rm) | 12.5 | 10.3 | 13.4 | 3.3 | 8.5 | 1.4 | — | 23.0 | — | — |
| Cvar(indiv) | 77 | 208 | 98 | 95 | 56 | 102 | — | 282 | — | — |
| Table 4: | | | | | | | | | | |
| DMI-derived immunomic data is shown for serum samples prepared from venous blood from 15 healthy donors (7 male and 8 female, aged 23 to 37) labelled ‘A’ to ‘O’. A single serum sample from another individual (male aged 35) was split into five replicate aliquots (P1 to P5) and also assayed. For each tag, the mean fluorescence bound is shown for pan-IgG (FITC) in the left-hand column and IgM (rPE) | | | | | | | | | | |
| # in the right-hand column. The variance components for each tag are broken down and presented: | | | | | | | | | | |
| ‘Cvar(anal)’ is the analytical variation from one tag to another within the same experiment. | | | | | | | | | | |
| ‘Cvar(rm)’ is the repeated measures variation for the 5 replicate aliquots, and is presented net of the analytical variation. | | | | | | | | | | |
| ‘Cvar(individ)’ is the individual-to-individual variation and is presented net of both analytical and repeated-measures variation. | | | | | | | | | | |
| Proteins with higher Cvar(individ) values contain the most diagnostic information. | | | | | | | | | | |
| Note that many of the tags yielded an approximately log-normal distribution, and that it would be appropriate log-transform the data prior to calculation of more accurate variance components. Furthermore, the data is heavily influenced by outliers - the impact of these outliers would be reduced by transformation, | | | | | | | | | | |
| # but Winzorising may be more appropriate once larger immunomic datasets were collected. | | | | | | | | | | |

The resulting vectors for the 15 individuals are shown in Table 4. For each antigen tag, there are two columns: the left-hand column contains the pan-IgG parameter and the right-hand column contains the IgM parameter. These vectors represent the IgG/M immunomic profile (focussed on carbohydrate antigens) for each of the individuals tested, and can be used for various investigational or analytical purposes.

In this example, we noted that about half the individuals had high levels of IgG (and also IgM) antibodies bound to tag 15 (values boxed in Table 4). This tag has the carbohydrate structure representing the A blood group antigen bound to it. The individuals with low levels of antibody must themselves express the A antigen and are either A or AB blood group. The individuals with high levels of antibody must not express the A antigen and are either 0 or B blood group. In fact, the same reasoning can be applied to the data from tag 16 which has the carbohydrate structure representing the B blood group antigen bound to it. From these two columns it is possible to determine that individual F is blood group A, while individual G is blood group B and individual L is blood group O. The same deductive process can be applied to all the individuals studied.

As for the use of DMI in proteomics (example 1), we have performed a fall analysis of the sources of variation within the immunomic dataset (Tables 3 & 4). Firstly, we have assessed the analytical reproducibility of the method (Cvar(anal)) calculated from the range of fluorescence readings from different tags with the same code in the same experiment. Unlike the proteomic analysis the Cvar(anal) varies from individual to individual because the absolute level of signal varies from individual to individual. The Cvar(anal) values reported are therefore the mean value for the 15 individuals studied. The analytical reproducibility is excellent (below 5% for most tags, superior to individual immunoassays).

Furthermore, five of the samples tested were replicate aliquots from the same bleed (P1 to P5, shaded in Table 4). This allows the repeated measures reproducibility (Cvar(rm)) to be assessed. The Cvar(rm) is reported with the analytical variation (Cvar(anal)) subtracted. The median Cvar(rm) for all 22 antigen tags for which a signal was detected in more than one test sample was 9% (range 0.8% to 49.5%) which is somewhat inferior to the application of DMI to proteomics. However, the reason for this lies in part in the very low signals which were obtained for many individuals on many of the tags—low signal, near the detection limit of the technique, is always detected with lower repeated measures reproducibility. However, the Cvar(individ), which represents the true individual-to-individual variance component is larger for the immunomic vectors than for the proteomic vectors (compare Table 4 with Table 2). This is the variance component which is useful for diagnostic modelling. Consequently, the true diagnostic utility of the test, which is approximated by Cvar(rm)/Cvar(individ) is very similar in the two applications of DMI.

It is important to note that the signal for each of the tags approximates a log-normal distribution, and that there are also a number of extreme outliers in the dataset. Consequently, a more thorough analysis would require log transformation (and possibly Winsorising) of the dataset prior to further investigation of the X-matrix.

### Example 4

### Preparation of a Large Peptide Antigen Library for DMI-Based Immunomics

To generate a large scale peptide antigen library, the following strategy was adopted: nine amino acid peptides were chosen to represent the master library. However, there are 20⁹ (about 5×10¹¹) sequence variants that compose this master library—many times too many for them all to be uniquely represented in the DMI antigen library. Therefore, to generate a library of manageable proportions, the amino acids were grouped into 4 groups of 5 based on similarity of properties (dominantly, charge and hydrophobicity). The groups selected were: GROUP 1 (charged) Arg, Lys, His, Asp, Glu; GROUP 2 (small hydrophobic) Gly, Ala, Leu, Ile, Val; GROUP 3 (large hydrophobic) Met, Phe, Pro, Tyr, Trp and GROUP 4 (hydrophilic) Ser, Thr, Asn, Gln, Cys. Alternative groupings could also be adopted, and would yield subtly different libraries that would still be suitable for immunomics. An equimolar mixture of the five amino acids within the group was then treated as a single reagent for combinatorial solid phase synthesis. There are, therefore, now just 4⁹ possible components to the library (262,144 components). Note, however, that each “component” is not a single peptide sequence but a mixture of 5⁹ (1.6 million) possible sequence variants—however, because of the grouping of the amino acids, related sequences are likely to fall within the same component pool.

The 262,144 component pools were synthesised by solid-phase synthesis using methods well known in the art. Briefly, each group of amino acids were coupled onto batches of solid phase resin. Each batch of coupled resin was then divided into four, and reacted with one of the four groups of amino acids, using appropriately protected amino acids. This process was then repeated, until a total of 262,144 batches of resin had been generated. Each was then cleaved and deprotected in parallel to yield 690 microtitre plates (384 wells per plate) each containing approximately 1 mg of peptide.

To each individual well, a different aluminium bar code tag pool was added appoximately 10⁶ identical individual tags in each case), and the peptide was allowed to bind to the tags. The tags were then removed and washed by gentle ultrafiltration, and resuspended in 100 μl of phosphate-buffered saline. All the components of the library were then combined, to yield 26 litres of pooled library containing approximately 10¹² individual tags (approximately 10⁷ tags per ml). This library was then concentrated by gentle ultrafiltration to a final volume of 250 ml (10⁸ tags/ml) which was then suitable for use at 20 μl per sample as in example 3 (allowing a total of more than 12,500 samples to be measured with this library.

This example demonstrates that it is possible to generate a very large antigen library capable of generating a high data density immunomic vector that contains information about antibodies recognising all possible 9 amino acid peptide antigens (every antigen is present, even though not every one is individually distinguishable as a separate library component). This library can be used to obtain an immunomic profile vector containing 2,359,296 individual datapoints for each individual in a procedure taking 30 minutes, exactly as described for the small carbohydrate antigen library in example 3.

### Example 5

### Use of DIM-Derived Immunomic Profiles to Diagnose Coronary Heart Disease

One purpose of deriving an immunomic profile using the DMI techniques described in this application is to obtain a high data density descriptive vector for different individuals which can be used to diagnose the presence of disease. This approach is exactly analagous to the use of genomics, transcriptomics, proteomics or metabonomics to make a diagnosis of a disease (for example, see Brindle et al. (2002) Nature Medicine 8:1439).

In the first step, a DMI-derived immunomic profile is obtained for a series of individuals whose disease status is known. In this example, we serum samples from 30 individuals, half known to have severe coronary artery disease (defined by angiography) and half with normal coronary arteries. These 30 individuals were a randomly chosen subset of the cohort of individuals described previously (Brindle et al. (2002) Nature Medicine 8:1439).

In the second step, pattern recognition methods are used to identify any signatures within the immunomic profiles which are uniquely and reproducibly associated with the presence of disease.

In a third step, the diagnostic power of the test is estimated by generating immunomic profiles from individuals whose disease status is not yet known, and making a prediction prior to determining the disease status using the gold-standard angiographic techniques.

A: Generating the Immunomic Profile

For this study, we elected to use an oligopeptide antigen library, composed of all possible octapeptide sequences (approximately 25 billion sequences). To reduce the library to a manageable number of entries, while retaining comprehensive sequence coverage, we adopted the principle described in Example 4 of preparing degenerate sub-libraries. Whereas a library made up of over 262,000 sub-libraries each containing almost 2 million sequences was described in Example 4, here we generated a simpler library made up of 256 sub-libraries each containing 100 million sequences. To do this, the 20 proteogenic amino acids were divided into just 2 groups as shown in Table 5, as opposed to the four groups used in Example 4.

TABLE 5

| Group 1 | Group 2 |
|---|---|
| INTERESTING (“I”) | BORING (“B”) |
| Arginine | Glycine |
| Lysine | Alanine |
| Histadine | Valine |
| Glutamate | Leucine |
| Aspartate | Isoleucine |
| Proline | Methionine |
| Cysteine | Asparagine |
| Serine | Phenylalanine |
| Threonine | Tyrosine |
| Tryptophan | Glutamine |

The library was then synthesised using standard solid phase synthetic chemistry, yielding approximately 50 mg of peptide in each sub-library. Each sub-library was then dissolved in 1 ml DMSO (to ensure equal dissolution of hydrophobic and hydrophilic sequences) and then diluted to yield a notional 10 mM stock solution (based on an average molecular weight of 880 for the octapeptides composing the library).

Immunomic profiles were then obtained in one of two different ways: (a) by solid phase immunoassay and (b) by multiplex solution assay.

To perform the solid phase immunoassay, the sub-libraries were individually diluted in 100 mM sodium carbonate pH 9.6 to yield 0.86 pmoles of peptide in 50 μl. High protein binding ELISA plates (Nunc Maxisorp) were then coated overnight with the diluted sub-libraries (264 wells, one coated with each sublibrary plus 8 additional wells coated with the sodium carbonate buffer alone composed a single experiment capable of measuring the immunomic profile of a single serum sample).

After coating, the solution was discarded by thoroughly aspirating the wells, which were then washed three times in wash buffer (Dulbecco's PBS containing 0.05% Tween 20). Non-specific binding was then blocked first by incubating the wells with 5% sucrose and 5% Tween 20 in Dulbecco's PBS (the first block buffer) then with 1% immunoglobulin-free bovine serum albumin in Dulbecco's PBS (the second block buffer). Wells were then washed a further 3 times.

The serum samples to be analysed were diluted 1:3.3 in second block buffer, and 100 μl was dispensed into each of the 264 coated wells. The sample was incubated in the wells for 2 hours at room temperature with shaking to allow antibodies in the serum to bind to the antigen sub-libraries. At the end of the incubation, the residual sample was discarded and the wells were washed five times to remove all unbound antibodies. The captured antibodies were then detected using a specific donkey antibody raised against human immunoglobulin-G (IgG), labelled with horseradish peroxidase (Jackson Immunoscientific). This antibody does not recognise any other class of human immunoglobulins, including IgM, and recognises the five IgG subclasses IgG1, IgG2a, IgG2b, IgG3 and IgG4) with approximately equal affinity. The detection antibody was diluted 1:5000 in second block buffer, and 200 μl was dispensed into each well. The plates were then incubated at room temperature, with shaking, for 1 hour.

At the end of this incubation, the detection antibody solution was discarded and the wells were washed three times. The amount of bound antibody was then quantitated by adding K-Blue (a horseradish peroxidase substrate), and measuring the amount of yellow product (after acidification) by spectrophotometry. The absorbance of the chromogenic substrate was proportional to the amount of IgG antibody in the serum sample which was able to bind to the particular sub-library of antigens. An immunomic profile was plotted by subtracting the average absorbance of the wells which were coated with sodium carbonate buffer only from each of the wells coated with sub-libraries, and then plotting the resulting net absorbance against sub-library number. In general, the hydrophilic sequences rich in I-group amino acids (Table 5) are in the lower-numbered sub-libraries to the left of the profile, while the more hydrophobic sequences rich in B-group amino acids (Table 5) are to the right of the profile.

To perform the solution phase multiplex assay, the sub-libraries were individually diluted in PBS to yield 86 pmoles of peptide in 500 μl. One million APTES-coated UltraPlex aluminium barcodes (SmartBead Limited) were pelleted by centrifugation (10,000×g; 10 secs) and then added to each sub-library, using a different barcode for each sub-library. The solutions were then incubated on a rotating shaker (which inverted the tubes approximately 10 times per minute) at 4° C. overnight.

After coating, the barcodes were pelleted using a filter-plate on a vacuum manifold and washed three times with wash buffer (Dulbecco's PBS containing 0.05% Tween 20). Non-specific binding was then blocked by incubating the barcodes with 1% immunoglobulin-free bovine serum albumin in Dulbecco's PBS (the block buffer) for 1 hour at room temperature. The barcodes were then washed a further 3 times. After the final wash, each sub-library was resuspended in 100 μl of PBS and all 256 sublibraries were combined to yield 25.6 ml of library solution. The library was then pelleted, and resuspended in 1 ml of PBS.

The serum samples to be analysed were dispensed, without dilution, at 200 μl per well in filter-bottom microtitre plates. 10 μl of library solution (being careful to ensure the barcoded elements were well mixed and thoroughly suspended in the 1 mL stock) was then added to each serum sample and the wells were incubated for 2 hours at room temperature on the rotating shaker to allow antibodies in the serum to bind to the antigen sub-libraries. At the end of the incubation, the library elements were pelleted and washed five times to remove all unbound antibodies using the vacuum manifold. The captured antibodies were then detected using a specific donkey antibody raised against human immunoglobulin-G (IgG), labelled with Alexa 488 fluorescent dye (Jackson Immunoscientific). This antibody does not recognise any other class of human immunoglobulins, including IgM, and recognises the five IgG subclasses (IgG1, IgG2a, IgG2b, IgG3 and IgG4) with approximately equal affinity. The detection antibody was diluted 1:500 in block buffer, and 200 μl was dispensed into each well. The plates were then incubated at room temperature, with shaking, for 1 hour.

At the end of this incubation, the library elements were pelleted and the wells were washed three times using the vacuum manifold. The amount of bound antibody was then quantitated using a fluoresence microscope to measure the amount of Alexa 488 fluoresence that was associated with each barcoded element. The fluoresence (in relative fluoresence units, RFUs) of at least 10 barcoded beads of each of the 256 sub-libvrary codes was measured, and the mean fluoresence was assumed to be proportional to the amount of IgG antibody in the serum sample which was able to bind to the particular sub-library of antigens. An immunomic profile was plotted by subtracting the average absorbance of the wells which were coated with sodium carbonate buffer only from each of the wells coated with sub-libraries, and then plotting the resulting net absorbance against sub-library number. In general, the hydrophilic sequences rich in I-group amino acids (Table 5) are in the lower-numbered sub-libraries to the left of the profile, while the more hydrophobic sequences rich in B-group amino acids (Table 5) are to the right of the profile.

A typical immunomic profile from an individual with coronary heart disease, upper left panel) and from an individual with normal coronary arteries (lower left panel) are shown in FIG. 5. The profiles shown were generated by the solid phase immunoassay method, but very similar profiles are obtained using the solution multiplex assay (r=0.742 across the 256 sub-library elements).

For most healthy individuals, there appears to be a prevalence of antibodies binding to the first 8 sub-libraries (which contain hydrophilic amino termina sequences rich in “I”-group amino acids), as well as to libraries in the range 120-180. On top of this “baseline” pattern, there are a number (about 10) individual sub-libraries which exhibit very strong signals (in some cases beyond the dynamic range of the assay). Preliminary analysis suggests that while the “baseline” pattern is relatively stable over time and between individuals, the “peaks” vary considerably, perhaps reflecting the specificities of the antibody clones which are currently expanded in response to pathogenic challenge.

B: Applying Pattern Recognition Methods

The immunomic profiles from 15 individuals with severe coronary artery disease and 15 individuals with normal coronary arteries were analysed for disease-specific patterns using Principal Component Analysis PCA). PCA is a megavariate statistical method ideally suited to the recognition of class-specific signatures in datasets with many more measured parameters (@) than observations (n). For our dataset (k=256, n=30), PCA revealed complete separation of the two groups in the first principal component (FIG. 5, right panel).

PCA is an unsupervised pattern recognition method (which means that the model shown in FIG. 5 was generated without knowledge of the disease status of any of the individuals) and is consequently robust to overfitting, and does not require external validation. It is possible to apply a supervised pattern recognition method (such as Partial Least Squares Discriminant Analysis, PLS-DA) which also yields excellent separation between the two groups. However, such models do require external validation, whereby profiles not used to generate the model are queried against the model. If the model is robust it correctly predicts these external validation profiles, while if the model is over-fitted the external prediction is substantially less good than the internal predictivity.

Using the PCA model shown in FIG. 5 it is possible to predict the disease status of individuals who have yet to undergo coronary angiography. The immunomic profile of the individual is obtained by the methods described in A: above, and that profile is used compared to the model shown in FIG. 5. Depending on the position of the new profile, we can make an unambiguous prediction of the disease status of the individual. Any of a number of methods well known in the art can be used to make such a prediction, such as a Cooman's Plot. The model shown in FIG. 5 has high positive and negative predictive value (estimated at >95%), such that it represents both a sensitive and a specific diagnostic test for the presence of coronary artery disease.

A range of other pattern recognition methods known in the art could be applied to the immunomic dataset we have generated here, including, but not limited to: genetic computing, support vector machines, linear discriminant analysis, variable selection algorithms and wavelet decomposition. In addition, a range of pre-processing filters known in the art could be applied to the data prior to application of the pattern recognition algorithm, including but not limited to: orthogonal signal correction, binning, adaptive binning, scaling and fourier transformation. In each case, it is necessary to determine by empirical application of the various available techniques, either together or in combination, which method yields the best separation between the immunomic profiles of the diseased and healthy individuals.

The method of the present invention, applying the use of immunomic profiles to the diagnosis of coronary artery disease is superior to existing methods to diagnose the disease. It is a non-invasive test, and therefore avoids the risk of complications and even death which accompany the gold-standard angiography test. It has considerably superior sensitivity and specificity compared with any existing uniparametric serum markers (such as cholesterol, LDL, HDL, triglyceride, CRP, fibrinogen or PAI-1) whether these measures are considered separately or together in a multi-parametric model such as the PROCAM model.

The method of the present invention is also superior to other high data density diagnostic platforms currently under development. Of these, the most sensitive and specific test described in the public domain is the NMR-based metabonomics test of Brindle and colleagues (Brindle et al. (2002) Nature Med. 8:1439). Although both the NMR-based test and the immunomics test of the present invention report >95% sensitivity and specificity, the separation between the two groups is greater in the immunomics dataset than in the metabonomics dataset, evidenced by the fact that complete separation of the two groups is only achieved in the metabonomics dataset after application of the Orthogonal Signal Correction filter to remove uncorrelated noise from the data matrix. No such application of OSC is required for the immunomics data matrix, which yields complete separation of the two groups in the first principal component of the unfiltered PCA model. This mathematical argument is fully supported by visual inspection: the immunomic profiles of the diseased individuals differ from those of the healthy individuals to a much greater extent than do the corresponding NMR-derived metabolic profiles (compare FIG. 5, left panel, with FIG. 1a in Brindle et al. (2002) Nature Med. 8:1439).

DMI-derived immunomics offers the further advantage of providing a diagnosis at a substantially lower cost that any of the other methods of comparable sensitivity and specificity (whether metabonomics, genomics, transcriptomics or proteomics). DMI-derived immunomics can be performed using the equipment present in a standard clinical diagnostic laboratory, using readily prepared reagents in contrast to metabonomics (which requires a specialised NMR spectrometer costing over £0.5 million), genomics (which requires gene-chip technology) and proteomics (which conventionally requires either 2D gel electrophoresis or liquid chromatography coupled with mass spectrometry).

1. A method of determining the relative abundance of a plurality of proteins in a test sample compared to a reference sample, the method comprising:
(a) providing a reference sample comprising a plurality of labelled proteins;
(b) incubating a plurality of tagged antibodies capable of binding components of the reference sample with (i) a mixture of the labelled reference sample and the test sample and (ii) the reference sample alone, under conditions suitable for the binding of said antibodies to their targets;
(c) comparing the amount of labelled protein bound to individual antibody tags in the presence and absence of the test sample.

2. A method according to claim 1 wherein said test sample and reference sample are mixed in equal volumes.

3. A method according to claim 1 wherein said antibodies are tagged with aluminium bar codes or dye impregnated beads

4. A method according to claim 1 wherein each tag is linked to a single antibody species.

5. A method according to claim 1 wherein each tag is linked to more than one species of antibody.

6. A method according to claim 5 wherein each of said antibody species linked to a tag binds the same protein.

7. A method according to claim 1 wherein each of said plurality of tagged antibodies binds a different protein.

8. A method according to claim 1 wherein from 10¹¹ to 10¹⁵ antibody molecules are bound to each tag.

9. A method according to claim 1 wherein said reference sample is obtained from the same tissue and/or organism as said test sample.

10. A method according to claim 1 wherein said reference sample is formed by pooling a plurality of test samples.

11. A method according to claim 1 wherein said proteins in the reference sample are labelled with one or more fluorescent dyes.

12. A method according to claim 1 wherein said binding is quantified by flow cytometry.

13. A mixture of peptides wherein each peptide is of length n amino acids and of the formula:
X₁—X₂—X₃— . . . —Xₙ
wherein:
each X represents an amino acid independently selected from one of a number of groups of amino acids;
each group of amino acids consists of less than 20 different amino acids;
n is the same for all peptides present in the mixture;
all of the following amino acids are present in at least one group: arginine, lysine, histidine, glutamate, aspartate, proline, cysteine, serine, threonine, tryptophan, glycine, alanine, valine, leucine, isoleucine, methionine, asparagine, phenylalanine, tyrosine and glutamine; and
for each peptide in the mixture the amino acid at the same position is selected from the same group.

14. A mixture of peptides according to claim 13 wherein no amino acid is present in more than one of said groups of amino acids and/or each group of amino acids contains the same number of different amino acids.

15. A mixture of peptides according to claim 14 wherein each X represents an amino acid independently selected from four groups of five amino acids or from two groups of ten amino acids and wherein no amino acid is present in more than one group.

16. A mixture of peptides according to claim 13 wherein each X represents an amino acid independently selected from one of two groups defined as follows:
(i) arginine, lysine, histidine, glutamate, aspartate, proline, cysteine, serine, threonine, tryptophan;
(ii) glycine, alanine, valine, leucine, isoleucine, methionine, asparagine, phenylalanine, tyrosine, glutamine.

17. A mixture of peptides according to claim 13 wherein n is 8.

18. A library comprising a plurality of mixtures as defined in claim 13 wherein each of said mixtures has the same value for n and the same groups of amino acids apply to all mixtures in the library, wherein (a) no peptide is present in more than one of said mixtures, and/or (b) the mixtures differ by virtue of the fact that the combination of groups chosen to obtain the peptides differs between the mixtures and optionally the library comprises mixtures representing all possible combinations of the groups.

19. A library according to claim 18 wherein each of said mixtures comprises a different tag.

20. A library according to claim 18 wherein said library comprises all possible peptides of length n.

21. A library according to claim 18 wherein the groups of amino acids are defined as follows:
(i) arginine, lysine, histidine, glutamate, aspartate, proline, cysteine, serine, threonine, tryptophan;
(ii) glycine, alanine, valine, leucine, isoleucine, methionine, asparagine, phenylalanine, tyrosine, glutamine.

22. A method of detecting a plurality of immunoglobulins in a test sample, the method comprising:
(a) providing a plurality of tagged antigens;
(b) incubating said tagged antigens of (a) with said test sample, under conditions suitable for the binding of any immunoglobulins present in said test sample to their targets;
(c) incubating said mixture of (b) with one or more labelled antibodies capable of binding specifically to immunoglobulins;
(d) measuring the amount of labelled antibody bound to each tagged antigen.

23. A method according to claim 22 wherein said plurality of antigens comprises oligopeptides and/or oligosaccharides.

24. A method according to claim 22 wherein each of said antigens comprises a different tag.

25. A method of claim 22 wherein said antigens are sub-divided into mixtures, each mixture comprising a different tag.

26. A method according to claim 25 wherein said antigens are peptides divided into mixtures on the basis of their amino acid sequence.

27. A method according to claim 26 wherein said mixtures are mixtures of peptides wherein each peptide is of length n amino acids and of the formula:
X₁—X₂—X₃— . . . Xₙ
wherein:
each X represents an amino acid independently selected from one of a number of groups of amino acids,
each group of amino acids consists of less than 20 different amino acids:
n is the same for all peptides present in the mixture;
all of the following amino acids are present in at least one group arginine, lysine, histidine, glutamate, aspartate, proline, cysteine, serine, threonine, tryptophan, glycine, alanine, valine, leucine, isoleucine, methionine, asparagine, phenylalanine, tyrosine and glutamine; and
for each peptide in the mixture the amino acid at the same position is selected from the same group.

28. A method according to claim 26 wherein said plurality of antigens is a library comprising a plurality of mixtures as defined in claim 13 wherein each of said mixtures has the same value for n and the same groups of amino acids apply to all mixtures in the library, wherein (a) no peptide is present in more than one of said mixtures, and/or (b) the mixtures differ by virtue of the fact that the combination of groups chosen to obtain the peptides differs between the mixtures and optionally the library comprises mixtures representing all possible combinations of the groups.

29. A method according to claim 22 wherein said labelled antibodies comprise antibodies specific to two or more immunoglobulin subclasses.

30. A method according to claim 29 wherein said antibodies specific to each immunoglobulin subclass comprise a different label.

31. A method according to claim 29 wherein said immunoglobulin subclasses are selected from IgG1, IgG2, IgG3, IgA, IgD, IgE and IgM.

32. A method according to claim 22 further comprising the step of quantifying the amount of each immunoglobulin subclass that binds each tagged antigen or tagged antigen mixture.

33. A method according to claim 22 wherein the amount of labelled antibody bound to each tagged antigen or tagged antigen mixture is measured by flow cytometry.

34. A method of detecting the presence of, or a susceptibility to, a disease or other medical condition comprising:
(i) detecting a plurality of immunoglobulins in a test sample obtained from an individual; and
(ii) comparing the immunoglobulins detected in the sample from said individual with known patterns of immunoglobulins associated with the presence or absence of a disease and thus determining whether said individual has, or is susceptible to said disease.

35. A method according to claim 34 wherein said patterns of immunoglobulins associated with disease are determined by a method comprising:
(i) detecting a plurality of immunoglobulins in test samples obtained from individuals whose disease status is known;
(ii) comparing the immunoglobulins detected between those individuals who are disease sufferers and those who are not and identifying any patterns associated with the presence or absence of the disease.

36. A method of detecting the presence of, or a susceptibility to, a disease or other medical condition comprising:
(i) detecting a plurality of immunoglobulins in test samples obtained from individuals whose disease status is known;
(ii) comparing the immunoglobulins detected between those individuals who are disease sufferers and those who are not and identifying any patterns associated with the presence or absence of the disease;
(iii) detecting a plurality of immunoglobulins in a test sample obtained from an individual by the same method used in part (i); and
(iv) comparing the immunoglobulins detected in the sample from said individual with the patterns identified in step (ii) and thus determining whether said individual has, or is susceptible to said disease.

37. A method according to claim 34 wherein said detecting is carried out by a method comprising:
(a) providing a plurality of tagged antigens;
(b) incubating said tagged antigens of (a) with said test sample, under conditions suitable for the binding of any immunoglobulins present in said test sample to their targets;
(c) incubating said mixture of (b) with one or more labelled antibodies capable of binding specifically to immunoglobulins;
(d) measuring the amount of labelled antibody bound to each tagged antigen.

38. A method according to claim 34 wherein said comparing is carried out using a pattern recognition method selected from Principal Component Analysis (PCA), Partial Least Squares Discriminant Analysis (PLS-DA), genetic computing, a support vector machine, linear discriminant analysis, variable selection algorithms and wavelet decomposition.

39. A method according to claim 34 which aids the diagnosis of a disease, aids the prediction of a future disease, aids the assessment of the severity of a disease, aids the monitoring of progression or regression of a disease or aids the monitoring of treatment of a disease in said individual.

40. A method according to claim 34 wherein said disease is coronary heart disease.

41. A kit suitable for use in a method of claim 22 said kit comprising
(i) a plurality of antigens or mixtures of antigens, wherein each antigen or mixture of antigens comprises a tag; and
(ii) one or more labelled antibodies capable of specifically binding to immunoglobulins.

42. A kit according to claim 41 wherein said plurality of antigens comprises oligopeptides and/or oligosaccharides.

43. A kit according to claim 41 wherein said labelled antibodies comprise antibodies specific to two or more immunoglobulin subclasses.

44. A kit according to claim 41 comprising:
(i) a library of peptides comprising a plurality of mixtures of peptides, wherein in each mixture, each peptide is of length n amino acids and of the formula:
X₁—X₂—X₃— . . . —Xₙ
wherein:
each X represents an amino acid independently selected from one of a number of groups of amino acids,
the groups of amino acids are defined as follows:
(a) arginine, lysine, histidine, glutamate, aspartate, proline, cysteine, serine, threonine, tryptophan;
(b) glycine, alanine, valine, leucine, isoleucine, methionine, asparagine, phenylalanine, tyrosine, glutamine;
n is the same for all peptides present in the mixture; and
for each peptide in the mixture the amino acid at the same position is selected from the same group:
wherein each of said mixtures has the same value for n and the same groups of amino acids apply to all mixtures in the library, wherein (a) no peptide is present in more than one of said mixtures, and/or (b) the mixtures differ by virtue of the fact that the combination of groups chosen to obtain the peptides differs between the mixtures and optionally the library comprises mixtures representing all possible combinations of the groups:
wherein each group of antigens is tagged with aluminium barcodes; and
(ii) a labelled antibody capable of specifically detecting human IgG.

45. A method of reducing the redundancy and bias of an antibody-expressing phage library comprising:
(a) providing two surfaces to which a sample of antigens is bound wherein said antigens are bound to the second surface at a higher density than to the first surface;
(b) exposing a phage display library to a first surface of (a) under conditions suitable for antibody binding and selecting phage bound to said surface;
(c) exposing said selected phage of (b) to a second surface of (a) under conditions suitable for antibody binding and selecting phage not bound to said surface;
(d) optionally further selecting said phage of (c) according to steps (b) and (c) one or more times;
thereby obtaining a library of antibody-expressing phage which has reduced redundancy and/or bias characteristics compared with the original library.

46. A method according to claim 1 wherein said plurality of antibodies is an antibody-expressing phage library produced by a method of reducing the redundancy and bias of an antibody-expressing phase library comprising:
(a) providing two surfaces to which a sample of antigens is bound wherein said antigens are bound to the second surface at a higher density than to the first surface;
(b) exposing a phage display library to a first surface of (a) under conditions suitable for antibody binding and selecting phase bound to said surface;
(c) exposing said selected phase of (b) to a second surface of (a) under conditions suitable for antibody binding and selecting phase not bound to said surface;
(d) optionally further selecting said phage of (c) according to steps (b) and (c) one or more times;
thereby obtaining a library of antibody-expressing phase which has reduced redundancy and/or bias characteristics compared with the original library.
